(19) United States
(12) Patent Application Publication
(10) Pub. No.: US 2020/0016088 A1
(43) Pub. Date: Jan. 16, 2020

(54) NANOFORMULATIONS OF NEUROPEPTIDES AND METHOD OF MAKING SAME

(71) Applicant: THE CORPORATION OF MERCER UNIVERSITY, Macon, GA (US)

(72) Inventors: Martin J. D'SOUZA, Duluth, GA (US); Kevin MURNANE, Norcross, GA (US)

(21) Appl. No.: 16/510,466

(22) Filed: Jul. 12, 2019

Related U.S. Application Data
(60) Provisional application No. 62/696,875, filed on Jul. 12, 2018.

(51) Int. Cl. A61K 9/51; A61K 38/095; A61K 9/00

(52) U.S. Cl. CPC A61K 9/5169; A61K 9/0043; A61K 38/095

(57) ABSTRACT

Compositions and methods for treating neurological disorders including Autism Spectrum disorder. The formulations include active compounds encapsulated in a nanoparticle, wherein the nanoparticle comprises a targeting ligand.

### CROSS-REFERENCE TO RELATED APPLICATION

This application claims benefit of co-pending U.S. provisional patent application No. 62/696,875, filed Jul. 12, 2018, entitled NANOFORMULATIONS OF NEUROPEPTIDES AND METHOD OF MAKING SAME, and commonly assigned to the assignee of the present application, the disclosure of which is incorporated by reference in its entirety herein

### GOVERNMENT SUPPORT CLAUSE

This invention was made with government support under NS 100512 awarded by the National Institutes of Health. The government has certain rights in the invention.

### FIELD

The present disclosure relates to compositions and methods to treat neurological disorders. In exemplary embodiments, compositions are disclosed that are designed to cross the blood-brain barrier and control the release of a compound.

### BACKGROUND

The development of new treatments for diseases of the central nervous system (CNS) is a pressing public health concern [1-4] (references in [brackets] refer to reference citations set forth hereinbelow). To treat neurological diseases in the brain, one route of treatment is delivering beneficial therapies, such as neuropeptides, across the blood-brain barrier (BBB) and into the brain. Neuropeptides are small protein-like molecules that are commonly co-released with neurotransmitters to modulate neurotransmission. Neuropeptides vary in their size, the neuroanatomical distribution of the neurons in which they are synthesized, and in the neurotransmitters with which they partner for co-release. They have shown particular promise as potential therapeutics for CNS disorders as observed in animal studies that demonstrate profound efficacy in treating a number of disease states. Although there are many neuropeptides with potential therapeutic benefits, one of particular interest is oxytocin (OT). OT is a nine-amino acid neuropeptide that is synthesized in the paraventricular and supraoptic nuclei of the hypothalamus. It is released into peripheral circulation by the posterior pituitary gland and also functions as a neurotransmitter throughout much of the mammalian brain [5]. Although OT has been traditionally recognized to be involved in uterine stimulation during parturition, recent studies have convincingly demonstrated its roles for the brain neurotransmitter OT system in social behavior, social recognition, social comfort, and altruism [6-8].

Unfortunately, a fundamental obstacle for the development of OT as a CNS medication is that it (1) does not readily passively cross the BBB following exogenous administration and (2) its half-life in the blood is short, on the order of minutes. The BBB limits large or hydrophilic molecules from readily entering the brain. This limits the range of compounds and scaffolds available to medicinal chemistry programs in the development of new chemical entities for treating CNS disorders. One method to overcome the resistance of the BBB is surgical cannulation of the brain ventricles for widespread or region-specific brain delivery of large molecule compounds to treat CNS disorders in animal models. Some of these compounds include neurotrophins such as brain-derived neurotropic factor (BDNF) [9-10], secretase inhibitors for Alzheimer's disease [11], anti-inflammatory cytokines [12-13], and therapeutic plasmid DNA for neuronal stimulation or myelin recovery and remodeling [14-16]. However, delivering these molecules by surgical cannulation is an invasive procedure. Alternatively, researchers have tried to utilize the mechanisms present in the BBB to deliver compounds to the brain without physical or surgical disruption of the barrier. Poor target penetrance and short half-lives are key factors that limit the ability to administer neuropeptides as therapies. A viable approach for large-molecule delivery to the CNS that does not require direct drug application would advance these compounds towards the clinic.

Receptor-mediated transport (RMT) [17] is a class of transport system that transports macromolecules such as insulin and transferrin between the blood and the brain. Transferrin (Tf) has been widely studied and shown to be a promising molecular probe for targeted drug delivery to the brain through RMT as transferrin receptors are known to be expressed in the luminal membrane of capillary endothelium of the BBB [18-20]. Tf is a single chain 80 kDa protein that facilitates the movement of iron between the blood and brain. Tf-bound iron from blood binds to the Tf receptor (TfR) expressed on the BBB. The TfR is expressed mostly on endothelial cells, intestinal cells, hepatocytes, and monocytes. Tf-conjugated drug delivery systems (nanoparticles, liposomes and micelles) can improve drug transport across the BBB since Tf-receptors (TfR) are overexpressed in brain capillary endothelium [21-26]. Previous studies have extensively examined the Transferrin (Tf) system as a mean of targeting drug formulations to the brain [22, 23, 27, 28]. Tf can be adsorbed on or conjugated to different polymers including PLGA, polyethylene glycol, lipopolyplexes, polymer-based cyclodextrin, and gold nanoparticles [29-32]. Recent studies have also described a new brain targeting ligand called Rabies Virus Glycoprotein (RVG), which putatively binds to nicotinic cholinergic receptors on the BBB to engage brain uptake of drug formulations [33]. Both Tf and RVG have been used previously to target therapeutic peptides to the brain, and are as such likely to be effective in the transport of neuropeptides into the brain.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Generally described, the present disclosure provides in a first exemplary embodiment a composition comprising an active material; a nanoparticle, wherein the nanoparticle encapsulates the active material; and, at least one targeting ligand, wherein the at least one targeting ligand is coupled to the nanoparticle.

In another embodiment, the composition comprises an active material, wherein the active is a neuropeptide.

In another embodiment the composition comprises an active material, wherein the active material is oxytocin.

In another embodiment the composition of comprises a nanoparticle, wherein the nanoparticle is comprised of BSA.

In another embodiment the composition comprises at least one targeting ligand, wherein the at least one targeting ligand is RVG. In another embodiment the RVG is adsorbed onto the nanoparticle.

The disclosure also generally includes a method of treating a neurological disorder comprising administering a composition described herein to a patient. In another embodiment the method includes treating a neurological disorder that is a central nervous system disorder. In another embodiment the neurological disorder is Autism Spectrum disorder.

In another embodiment the composition to treat a neurological disorder is administered intranasally.

Other features will become apparent upon reading the following detailed description of certain exemplary embodiments, when taken in conjunction with the appended claims.

### BRIEF DESCRIPTION OF THE DRAWINGS

The drawings disclose exemplary embodiments in which like reference characters designate the same or similar parts throughout the figures of which:

FIGS. 1A-1C are graphs of representative particle sizes and morphology of the nanoparticle formulations. FIG. 1A shows size distribution of PLGA nanoparticles conjugated to Tf or RVG.

FIG. 1B shows size distribution of BSA nanoparticles conjugated to Tf or RVG. FIG. 1C shows scanning electron microscopy images of the BSA nanoparticles conjugated to Tf at a 3 um resolution (c). The BSA nanoparticles conjugated to RVG were too small to image at a 3 um resolution. Tf=Transferrin; RVG=Rabies Virus Glycoprotein; PLGA=poly(lactic-co-glycolic acid); BSA=Bovine serum albumin; OT=Oxytocin.

FIGS. 2A-2D are graphs of FT-IR spectrum data collected to confirm the conjugation of the brain targeting ligands to the nanoparticles. FIG. 2A shows a FT-IR spectrum of Tf conjugated to PLGA nanoparticles. Two main peaks can be seen at 1750 cm-1 and 1648 cm-1 that can be assigned to the amide I and amide II vibrations, respectively. FT-IR spectrum of blank and unconjugated PLGA nanoparticles shows characteristic bands of carbonyl C═O stretching (1750 cm-1) and the ester C—O stretching (1000-1300 cm-1). FT-IR spectrum of PLGA nanoparticles conjugated to Tf shows the amide bands assigned to the protein. The peak of the amide I band in the spectrum of PLGA-Tf nanoparticles occurs at 1648 cm-1. The bands of the characteristic functional groups of PLGA nanoparticles are present (1750 cm-1 and 1000-1300 cm-1) with small shifts when compared to the spectrum of the unmodified nanoparticles. The presence of amide I band in the PLGA-Tf spectrum demonstrates that Tf has been conjugated to the PLGA nanoparticles. FIG. 2B shows a FT-IR spectrum of RVG conjugated to PLGA nanoparticles. FIG. 2C shows a FT-IR spectrum of Tf conjugated to BSA nanoparticles. FIG. 2D shows a FT-IR spectrum of RVG conjugated to BSA nanoparticles. Both BSA spectra show two main peaks near 1650 cm-1 and 1540 cm-1, which are the two peaks of the amide I and amide II vibrations of Tf and RVG, indicating conjugation of RVG and Tf to BSA.

FIG. 3 is a table showing the induction of nitric oxide release by the lead oxytocin formulations. The oxytocin formulation was generated by double emulsion solvent evaporation by sonication. All points represent the mean±standard error. Abscissae: Various formulations compared to the negative control of untreated cells and the positive control of the 4T1 breast cancer vaccine that induces a robust immune response. Ordinates: Measured concentration of nitric oxide expressed on an absolute scale in units of micromolar.

FIG. 4 is a cytotoxicity profile of the nanoparticle formulations. After 48 hours of exposure, none of the four formulations were cytotoxic across the tested concentration range (0.0625-1 mg/mL). As there were no differences between the concentrations tested, only the highest concentration (1 mg/mL) is shown. Abscissae: Various formulations compared to the negative control of untreated cells and the positive control of benzalkonium chloride (a cationic surfactant that has biocidal activity). Ordinates: Cell viability expressed as a percentage of the negative control at 6 hours. All experiments were conducted in triplicate.

FIG. 5 show the cumulative release of OT from PLGA and BSA nanoparticles in PBS, pH 7.4. The release of OT from PLGA nanoparticles was sustained over a period of 21 days. OT was released from PLGA nanoparticles in sustained fashion with 50% of the OT released over a period of 10.8 days. The release of OT from BSA nanoparticles was sustained over a period of 18 days and 50% of OT was released over a period of 9.8 days. Abscissae: Day following addition of the formulation to solution. Ordinates: Measured concentration of free oxytocin in solution expressed as a percentage of the maximally measured release. PLGA=poly(lactic-co-glycolic acid); BSA=Bovine serum albumin; OT=Oxytocin.

FIG. 6 shows the release of OT from PLGA (prepared by sonication) and BSA nanoparticles in 10% serum, pH 7.4 over 6 hours at 4° C. and 37° C. At the end of 6 hours, 37.07% and 32.8% of the OT has been released from the BSA nanoparticles at 4° C. and 37° C. temperature, respectively. In contrast, only 5% of the OT had been released from the PLGA nanoparticles at either temperature. Abscissae: Hour following addition of the formulation to solution. Ordinates: Measured concentration of free oxytocin in solution expressed as a percentage of the maximally measured release. PLGA=poly(lactic-co-glycolic acid); BSA=Bovine serum albumin; OT=Oxytocin.

FIG. 7 shows the release of OT from BSA nanoparticles in 10% serum, pH 7.4 at 4° C. 50% of the OT was released over a period of 3 days. Abscissae: Day following addition of the formulation to solution. Ordinates: Measured concentration of free oxytocin in solution expressed as a percentage of the maximally measured release. BSA=Bovine serum albumin; OT=Oxytocin.

FIGS. 8A and 8B show validation studies to determine the integrity of the artificial blood-brain barrier formed by BEND3 cells. FIG. 8A shows a representative photomicrograph of monolayer and tight junction formation by BEND3 cells. FIG. 8B shows the barrier integrity was confirmed by examining transfer of trypan blue dye across the membrane using spectrophotometry at 540 nm. All points represent the mean±SEM. Abscissae: Hours following the introduction of the Trypan blue dye to the left compartment of the Ussing chamber. Ordinates: Percentage of the dye that crossed to the right side of the Ussing chamber normalized to the amount remaining on the left side. All assessments were conducted in triplicate. *=p<0.05 in comparison to the presence of the cellular barrier.

FIG. 9 shows transport of fluorescein isothiocyanate (FITC) encapsulated in Tf-coated nanoparticles across the artificial blood-brain barrier formed by BEND3 cells. The extent of transport was assessed by quantifying the fluorescence intensity of FITC using a fluorescent spectrophotometer at an excitation wavelength of 485 nm and an emission wavelength of 528 nm. All points represent the mean±the standard error of the mean. Abscissae: Hours following the introduction of the FITC dye to the left compartment of the Ussing chamber. Ordinates: Percentage of the dye that crossed to the right side of the Ussing chamber normalized to the amount remaining on the left side. All assessments were conducted in triplicate.

FIGS. 10A and 10B show the extent of uptake indocyanine green (ICG) dye in the brain when given alone or in either transferrin (Tf) or rabies virus glycoprotein (RVG)-coated nanoparticles through intranasal administration in Swiss-Webster mice. The integrated intensity of the dye in the brain was determined using a LI-COR Odyssey bioimager at 800 nm. TOP: Time course of ICG uptake in brain. BOTTOM: Area under the curve determined across the individual mice. Abscissae: Hours following intranasal administration of ICG (FIG. 10A) or treatment condition (FIG. 10B). Ordinates: Integrated uptake of ICG in brain expressed in raw values (FIG. 10A) or as the area under the curve (FIG. 10B). All assessments were conducted in triplicate. *=p<0.05 as assessed by one-way analysis of variance and Dunnett's post-hoc test in comparison to ICG alone.

FIGS. 11A-11B shows concentration of oxytocin (OT) in cerebrospinal fluid (CSF) 2 hours after intranasal administration of OT alone or OT encapsulated in rabies virus glycoprotein (RVG)-coated nanoparticles. FIG. 11A shows the concentration of OT in the CSF. FIG. 11B shows the percentage of OT in the CSF determined in proportion to the total concentration of OT in CSF and plasma. OT levels were determined in CSF and plasma by ELISA. Abscissae: Treatment condition. Ordinates: Concentration of OT in CSF (FIG. 11A) or percentage of OT in CSF compared to CSF plus plasma (FIG. 11B). All assessments were conducted in duplicate. *=p<0.05 as assessed by unpaired t-test.

FIGS. 12A and 12B shows dyadic social interactions following delivery of oxytocin (OT) alone or in either transferrin (Tf) or rabies virus glycoprotein (RVG)-coated nanoparticles through intranasal administration in Swiss-Webster mice. Social interactions were assessed over a period of 10 minutes and were observationally scored using 6-point scale. FIG. 12A shows the social behavior assessed 2 hours after OT administration. FIG. 12B shows the social behavior assessed 3 days after OT administration. Abscissae: Treatment condition. Ordinates: Time in seconds spent in any social interaction. *=p<0.05 compared to vehicle; **=p<0.01 compared to vehicle; #=p<0.05 compared to oxytocin alone as assessed by one-way analysis of variance and Tukeys's post-hoc test.

FIGS. 13A and 13B shows an assessment of the general investigation component of dyadic social interactions following delivery of oxytocin (OT) alone or in either transferrin (Tf)- or rabies virus glycoprotein (RVG)-coated nanoparticles through intranasal administration in Swiss-Webster mice. FIG. 13A shows the social behavior assessed 2 hours after OT administration. FIG. 13B shows the social behavior assessed 3 days after OT administration. Abscissae: Treatment condition. Ordinates: Time in seconds spent in general investigation. *=p<0.05 compared to vehicle; **=p<0.01 compared to vehicle; #=p<0.05 compared to oxytocin alone as assessed by one-way analysis of variance and Tukeys's post-hoc test.

### DETAILED DESCRIPTION

Compositions and methods are disclosed for targeting, penetrating, or crossing the blood-brain barrier (BBB). Further, compositions and methods are disclosed for treating a neurological disorder in a patient.

In one exemplary embodiment, a composition is administered to a patient to treat a neurological disorder. In accordance with the disclosure, “patient” may refer to a human or an animal. Accordingly, the methods and compositions disclosed herein can be used, or adapted for use with both human clinical medicine and veterinary application.

In one exemplary embodiment, an active material is encapsulated in at least one nanoparticle, wherein the nanoparticle is coated in targeting ligands.

In one exemplary embodiment, nanoparticles can be used to encapsulate an active material. Further, the active material can be a target neuropeptide and the nanoparticles encapsulating the target neuropeptide can be coated with a targeting ligand (e.g., Tf or RVG) to utilize receptor-mediated transport. One type of receptor-mediated transport is referred to as transcytosis and can be used to move an active material across the BBB. Receptor mediated transcytosis is a class of transport system that transports macromolecules like insulin and Tf between blood and brain. Nanoparticles having sizes smaller than 500 nm are usually taken via the clathrin-mediated transport system whereas larger particles are usually transported via micropinocytosis [54]. In reference to charge, negatively-charged nanoparticles are endocytosed by interacting with the positive site of the proteins on a membrane.

In another exemplary embodiment, the active material can be encapsulated within different polymers including poly(lactic-co-glycolic acid) (PLGA), Bovine serum albumin (BSA), polyethylene glycol, lipopolyplexes, polymer-based cyclodextrin, and gold nanoparticles [29-32]. Additionally, smaller nanoparticles (e.g., under 200 nm in diameter) can potentially increase the penetrance of the formulation through the BBB and into the brain. In further exemplary embodiments, the nanoparticles can range in an average size of up to 200 nm in diameter. In other exemplary embodiments, the nanoparticles can range in an average size of less than 200 nm in diameter. In other exemplary embodiments the nanoparticles' diameters can range in an average size of about 20 nm, about 30 nm, about 40 nm, about 50 nm, about 60 nm, about 65 nm, about 70 nm, about 75 nm, about 80 nm, about 85 nm, about 90 nm, about 95 nm, about 100 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, and about 210 nm. Furthermore, both PLGA and BSA nanoparticles have sustained release properties, which can increase the half-life of an active material (e.g., oxytocin (OT)) and thereby decrease the frequency of administration, which can increase patient compliance. In other exemplary embodiments, a composition comprising PLGA or BSA nanoparticles have a release profile that provides an initial burst release of an active material (e.g., a peptide) to induce rapid symptom control followed by a sustained release for prolonged therapeutic effects.

In one exemplary embodiment the active material include can one or more of the following: small molecules, biologics, peptides, neuropeptides, macromolecules, antibodies or fragments thereof, ligands, DNA, RNA, proteins, fluorescent markers, or a combination or mixture of at least two of the foregoing. In certain exemplary embodiments, a neuropeptide is disclosed. In further exemplary embodiments, the neuropeptide is oxytocin (OT).

In another exemplary embodiment, the composition is used to treat a neural disorder. In some embodiments the neural disorder can be a central nervous system (CNS) disorder with social deficit components, including Autism Spectrum Disorder (ASD) and Dravet syndrome.

In one exemplary embodiment, two polymeric-based nanoparticle formulations were developed in order to increase the brain penetrance and extend the duration of action of OT.

For the first formulation, the OT-loaded PLGA particles were prepared by dissolving poly(lactic-co-glycolic acid) (PLGA) in dichloromethane, adding the OT along with sorbitan monooleate, homogenizing the particles in a water/oil emulsion, and adding poly vinyl alcohol to create a final water/oil/water (w/o/w) emulsion.

The second formulation, OT-loaded BSA-crosslinked particles were created by a coacervation/nanoprecipitation technique (see further details in the Examples hereinbelow; all references to “Examples” refer to the Examples described hereinbelow) to encapsulate OT and form the particles.

Next, the physical, physiochemical, and release properties of the formulations were determined as well as their potential for undesirable immunogenic reactions. Also, formulations composed of PLGA and BSA particles conjugated with transferrin (Tf) or rabies virus glycoprotein (RVG) were compared in order to select a formulation based on the criteria for a desired particle size and release profile. Of the four final formulations, the OT-loaded BSA nanoparticles conjugated to RVG were found to produce the smallest-sized particles and maintained the desired release profile of OT. In further embodiments, the composition comprised Tf-PLGA, Tf-BSA, RVG-PLGA, or RVG-BSA. These four formulations were formulated and characterized (see further details in the Examples hereinbelow). In one exemplary embodiment, a release profile provides an initial burst release of the active material (e.g., a peptide) followed by a sustained release. This is one exemplary profile for therapeutic effects, as well as the profile for in vivo testing. The data show that BSA particles conjugated to RVG offer useful properties.

Receptor-mediated transport (RMT) is a useful strategy for delivering drugs to the brain [17] and targeting ligands can be chosen or designed based on their ability interact with receptors on neural cells and transport a composition into the brain across the BBB. In one exemplary embodiment, transferrin (Tf) was coupled to the nanoparticles of the composition wherein the Tf was able to interact with the transferrin receptors expressed on the luminal membrane of capillary endothelium of the BBB [18-20]. In another exemplary embodiment RVG is coupled to the nanoparticles of the composition. Rabies virus glycoprotein (RVG) is a short peptide that can bind to nicotinic cholinergic receptors on the BBB and it has been employed as a targeting ligand to deliver small interfering RNA (siRNA) to the brain [48, 49]. In another exemplary embodiment, both Tf and RVG are coupled to the nanoparticles of the composition. In another exemplary embodiment the composition comprises two different groups of nanoparticles wherein the first group has Tf coupled to the first set of nanoparticles and the second group of nanoparticles has RVG coupled to the second set of nanoparticles. Both the first and second set comprise the composition. The first set may be administered first to a patient followed by the second set of nanoparticles. Alternatively, the first and second set of nanoparticles may be administered at the same time. With the provided new formulation and methods, compounds with potentially therapeutic use for CNS disorders that have been abandoned because of poor brain bioavailability can now be useful. Some of these compounds include neurotrophins such as BDNF [9, 10], secretase inhibitors for Alzheimer's disease [11], anti-inflammatory cytokines [12, 13], and therapeutic plasmid DNA for neuronal stimulation or myelin recovery and remodeling [14-16]. Neuropeptides such as neuropeptide Y, BDNF, orexin, and others have defined disease states where they show profound therapeutic promise, including epilepsy, substance-use disorders, and obesity.

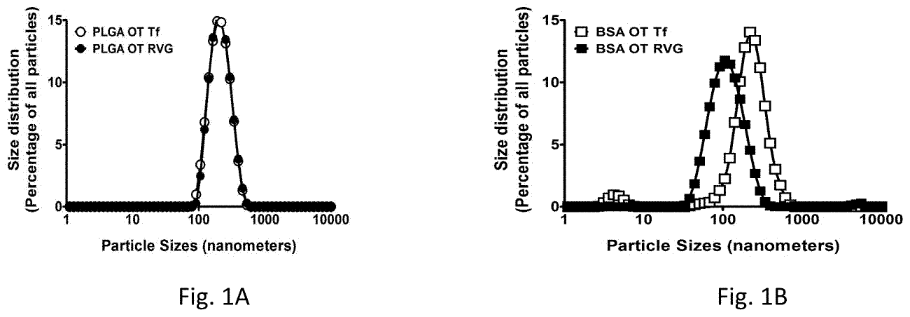

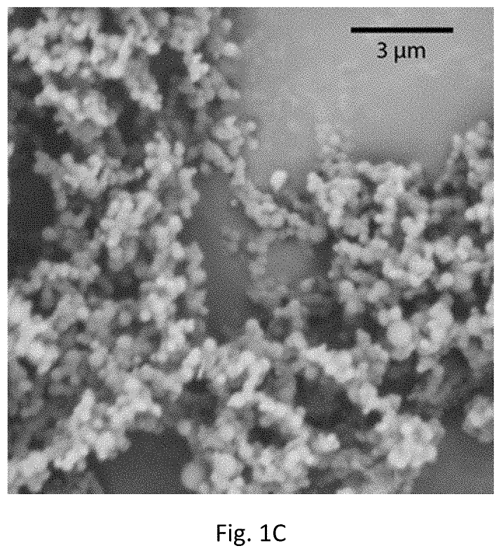

The size, shape, and charge of the particles are important factors to consider for delivering drugs across the BBB [53]. It has been reported in a previous study that the negatively charged nanoparticles are highly endocytosed by cells due to their repulsive interactions with the negatively charged cell surface [54, 55], and positively charged particles show substantially reduce BBB penetrance. The nanoparticle formulations exhibit a smooth, regular and spherical shape, which helps maximize uptake, as spherical particles are efficiently taken up via both clathrin and caveolae mediated transport (see FIG. 1) [56, 57]. In certain exemplary embodiments, it may be desirable to have particles below 200 nm as this will maximize the probability of efficient brain uptake. In initial studies, PLGA particles conjugated to Tf were generated using homogenization. However, the particles size exceeded 200 nm with this approach. We modified the production method to sonication and were able to readily achieve particles sizes below 200 nm. Such sizes were also readily achievable with BSA nanoparticles. However, it is important to note the BSA particles conjugated to RVG clearly achieved the smallest particles sizes.

To demonstrate the therapeutic viability of nanoparticulate formulations of certain exemplary embodiments of the present disclosure, both immunogenicity and cytotoxicity studies in vitro were completed. (See Examples 10 through 16 hereinbelow). As confirmed in past research, the nanoparticle formulations have shown to be both safe and non-toxic in non-human primates. In primates, all parameters related to liver toxicity (such as SGOT, SGPT) and kidney toxicity (such as serum creatinine, serum bilirubin) were normal after repeated nanoparticulate administrations when compared to the control group. Also, no antibody titers in response to the polymeric particle matrix were detected demonstrating the absence of an immune response to the polymer matrix formulation [36]. For confirmation of a potential innate inflammatory response, nitric oxide assays are an important tool. For the assay, dendritic cells are plated in a well plate and exposed to the formulation. After 16 hours, using reagents, a stronger color change indicates a higher production of nitric oxide by these antigen-presenting cells in response to the formulation's antigenicity. No significant difference in the amount of nitric oxide released in the supernatant of cells exposed to peptide-loaded nanoparticles compared to blank nanoparticles or the cells alone was demonstrated. This indicates minimal if not negligible immunogenicity of the formulation. For reference, nitric oxide release in response to the OT-loaded particles was substantially lower compared to cells that were exposed to a well-known highly-immunogenic breast cancer vaccine formulation. Likewise, cytotoxicity of blank nanoparticles was assessed by MTT (3-(4,5-Dimethylthiazol-2-yl)-2,5-diphenyltetrazolium bromide) assay in a macrophage (RAW 246.7) cell line. No significant cell death following 48 hours of exposure to the formulations were found indicating that the formulation was not significantly toxic to the cells.

In one exemplary embodiment the nanoparticle formulation of compositions for peptide delivery protects the encapsulated peptide from degradation, and sustains the delivery of the therapeutic agent over an extended period of time. This sustained delivery allows for fewer administrations and easier compliance, an issue of particular importance in the treatment of substance abuse disorders for a prolonged amount of time or in cases where patients have difficulty with compliance. Resultantly, the active material (e.g., OT) can provide long-lasting therapeutic effects in the brain, improving the health of those suffering with various cognitive ailments.

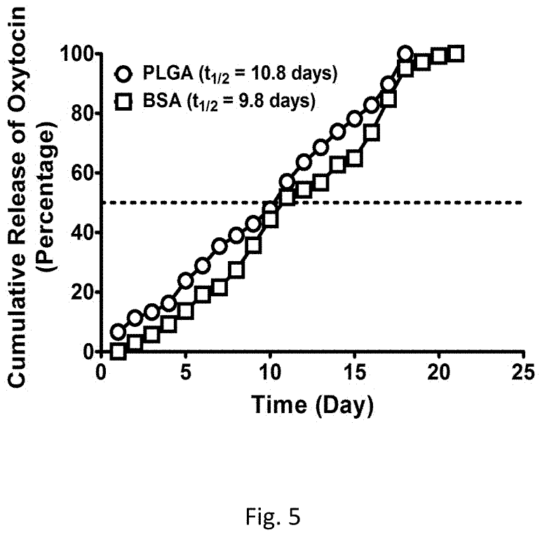

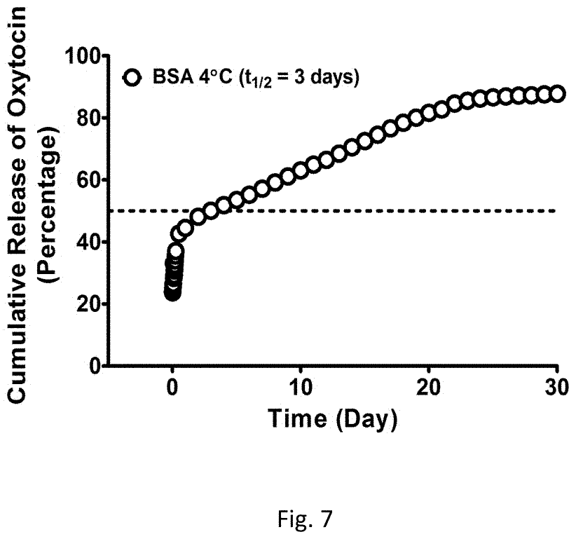

In one exemplary embodiment, the compositions (also referred to herein as formulations) maintain a sustained release profile of OT in vitro in both PBS and plasma, which was confirmed using ELISA (FIGS. 5 and 7). During the release studies, samples were taken in 30-minute interval initially, then hourly, and then daily to establish a release profile covering several weeks. In order to select the most viable formulation to test in vivo, two factors were explored: size and suitable sustained release criteria. The RVG-conjugated BSA nanoparticles were found to be smaller than the PLGA particles or Tf-conjugated BSA particles. The rank order of size of the nanoparticulate formulations was as follows: RVG-conjugated BSA (100.1 nm), Tf-conjugated BSA (196.3 nm), Tf-conjugated PLGA (191.7 nm), RVG-conjugated PLGA (201.2 nm). For the purpose of the treatment with OT, an initial burst release followed by a sustained release for several days was desired. Hence, it was determined that the PLGA nanoparticle formulations would not be viable candidates since they released OT very slowly over many days. The OT release in 6 hours in 37 degrees Celsius serum was as follows: BSA nanoparticles (32.8% release), and PLGA nanoparticles (5% release). Hence, such a slow release profile would reduce the therapeutic window for OT in the brain. Hence, when selecting the final formulation to test in vivo, it was determined that the BSA nanoparticles conjugated to RVG formulation was the most suitable candidate. Although the two BSA-based formulations demonstrated desirable release profiles, the RVG-conjugated BSA microparticles were almost half the size of Tf-conjugated BSA microparticles. Hence through taking into account size and the OT-release profile, it was concluded that RVG-conjugated BSA nanoparticles would be the most appropriate formulation to move forward with in the in vivo murine models. (See Examples 10 through 20).

In the example described hereinabove, biodegradable Tf or RVG-conjugated PLGA and BSA nanoparticles loaded with oxytocin were successfully prepared by solvent evaporation and nanoprecipitation/coacervation methods respectively. The formulation was also characterized using various methods such as zeta sizing and scanning electron microscopy (See Examples). Significant parameters affecting particle size were ranked and optimized to produce the final formulations for in vivo testing. The nanoparticles produced were uniformly distributed in nanometer range, spherically shaped, and individually dispersed. The nanoparticles formulations were non-immunogenic and non-toxic. All four formulations showed sustained delivery by sustained release of the therapeutic agent allowing for fewer administrations and easier compliance. Therefore, the nanoparticulate formulation could serve as a potential candidate for delivering drugs and therapeutic peptides across the BBB non-invasively.

In one exemplary embodiment, a nanoparticle drug delivery system was developed and evaluated for OT designed to increase its brain bioavailability through active transport and sustain its delivery through encapsulation and sustained release. First, transport of OT-like large molecules using the technology in a cell culture model of the BBB was evaluated. Then, in vivo brain transport using bioimaging and cerebrospinal fluid (CSF) analysis was determined. Finally, the efficacy of OT in two different brain targeting and sustained-release formulations in a mouse model of social behavior was determined. One hypothesis is that this novel drug formulation increases the brain penetrance and sustains the delivery of intranasal OT.

Intranasal delivery is favorable when adverse or other effects limit oral dose delivery or when increasing brain penetrance is desirable. In one exemplary embodiment, the composition is provided to a patient intranasally. In this embodiment, the composition comprising an active material encapsulated by at least one nanoparticle and at least one targeting ligand coupled to the at least one nanoparticle, wherein the targeting ligand is chosen or designed because of its ability to interact with the luminal membrane of the BBB. The composition is administered intranasally to a patient wherein the composition crosses the BBB. In one exemplary embodiment, the composition is provided intranasally to treat a neurological disorder. In another embodiment the composition is provided in a liquid. The composition liquid mixture is aerosolized through a device to generate a mist of the composition liquid that is inhaled by a patient. The device can be any suitable aerosolizing device known to those skilled in the art.

In another exemplary embodiment, the composition comprises an active material, at least one nanoparticle to encapsulate the active compound, and a targeting ligand coupled to the at least one nanoparticle. The targeting ligand can be RVG. Further, the composition comprising the RVG targeting ligand is provided in a liquid. The liquid is aerosolized using a device known to those having ordinary skill in the art. The device aerosolizes the composition liquid creating a mist for a patient to inhale. The RVG targeting ligand interacts with nicotinic cholinergic receptors (nAChR) on the BBB to engage brain uptake of drug formulations (Kim et al. 2013; Liu et al. 2016). nAChR subunits are expressed in the nasal mucosa, including alpha and beta subunits (Keiger et al. 2003). There is evidence that nAChRs are expressed at significant densities on nasal trigeminal nerve endings of the rat, suggesting an anatomical distribution of these receptors that may facilitate direct bypass of the BBB (Alimohammadi and Silver 2000). Intranasal inoculation of the rabies virus is believed to allow the virus to directly access the brain via the olfactory epithelium, either along the olfactory nerve or the trigeminal nerve (Lafay et al. 1991; Terryn et al. 2014). In addition to RVG, there are a number of other receptor and transporter systems that allow for the development of novel brain targeting nanoparticle formulations. For example, the P-glycoprotein transporter, excitatory amino acid transporter, dopamine transporter, amino acid transporters, and nucleoside transporters among others are expressed in the nasal mucosa (Anand et al. 2014).

It is well known that OT alone is rapidly metabolized in both blood and cerebrospinal fluid (CSF). OT activates a plethora of systems in the periphery, most notably in the uterus, but in other organ systems as well. It is therefore important that intranasal OT should achieve the best possible ratio of brain to blood levels. Previous work in rodents, primates, and humans indicates that the doses required to achieve brain penetration may induce supraphysiological effects in the periphery (Born et al. 2002; Dal Monte et al. 2014; Leng and Ludwig 2016; Modi et al. 2014; Robertson et al. 1970; Striepens et al. 2013). This problem may not be addressable without a brain penetrance enhancer, as based on these studies, the calculated maximum transfer of a dose of OT to the brain within one hour of intranasal delivery is 0.005% (Leng and Ludwig 2016). Moreover, OT concentrations are basally higher in CSF than plasma, and the plasma spillover of intranasal OT thus drives a much higher proportional change in plasma than CSF. The composition and methods of use disclosed herein provide at least two significant advantages over intranasal delivery of OT alone. First, the release of the peptide is sustained and therefore likely limits the potential for repeated bolus activation of peripheral systems following the frequent dosing that would likely be required for intranasal OT in the absence of a sustained release formulation. Second, a greater proportion of the OT to the brain is targeted, both during the initial application and during any subsequent passes in the blood circulation that contacts the BBB, which increases the proportion in the brain relative to the periphery and may reduce the required dose. The in vitro and in vivo data support these contentions.

The transport of OT-like large molecules using the functionalized nanoparticle technology in a cell culture model of the BBB and in vivo using bioimaging was evaluated. The efficacy of OT in two different brain targeting and sustained-release formulations in a mouse model of social behavior was determined. It was found that the nanoparticle formulation of certain exemplary embodiments of the present disclosure increases BBB transport both in vitro and in vivo. Moreover, nanoparticle-encapsulated OT administered intranasally exhibited greater pro-social effects both acutely and 3 days after administration relative to OT alone. Both Tf and RVG nanoparticles produced robust acute and sustained elevations in overall social behavior that were comparable in their magnitude to each other. These acute pro-social effects were also in near agreement in their magnitude to those of MDMA in mice (Curry et al. 2018) and MDMA in rats (Morley et al. 2005; Thompson et al. 2007), which is a strongly prosocial and brain penetrant small molecule that elicits release of OT (Harris et al. 2002; Murnane et al. 2010; Murnane et al. 2012; Thompson et al. 2007). Interestingly, the two different brain targeting ligands differed in the nature of the pro-social effects that they elicited, with RVG selectively increasing general investigation and Tf increasing a mixed response of general investigation in some subjects and adjacent lying (data not shown) in other subjects with acute administration. Previous studies have shown that acute MDMA administration selectively increases general investigation in mice (Curry et al. 2018) but has more of a mixed response between general investigation and adjacent lying in rats (Morley et al. 2005; Thompson et al. 2007). It is possible that the differences between these two brain targeting ligands may be due to the kinetics of brain transport as mice that were treated with OT in nanoparticles conjugated to either ligand showed selective increases in general investigation 3 days after administration. These multimodal data strongly support the use of the approach to develop a brain targeting and sustained-release formulation of OT. This formulation can now be used to support intranasal delivery of OT and other neuropeptides.

The following examples are set forth for purposes of illustration only. Parts and percentages appearing in such examples are by weight unless otherwise stipulated.

### EXAMPLES

### Example 1: Materials

Poly(D,L-lactide-co-glycolide) lactide:glycolide (50:50), polyvinyl alcohol, dichloromethane, indocyanine green, transferrin (Tf), and benzalkonium chloride were purchased from Sigma Aldrich (Milwaukee, Wis., USA). Bovine serum albumin (Fraction V), glutaraldehyde (25% in water) and acetone were purchased from Fischer Scientific (Pittsburgh, Pa., USA). Rabies Virus Glycoprotein (RVG) was purchased from GenScript (Piscataway, N.J., USA). All the chemicals were of analytical grade and used as is without further modification.

### Example 2: Experimental Design

A full factorial design to optimize the parameters for the BSA nanoparticle formulation such as pH of the aqueous solution, polymer concentration, and antisolvent (acetone): solvent (water) ratio was performed. All three variables were evaluated at three different levels using a full factorial design of experiments. Results of the experiments were analyzed using JMP® (SAS) software as previously demonstrated by the group [37-39].

### Example 3: Preparation of Nanoparticles

PLGA nanoparticles were prepared using a multiple emulsion solvent evaporation and lyophilization method. Briefly, 100 mg of PLGA and 20 μl of sorbitan monooleate were dissolved in 5 ml dichloromethane. 10 mg of OT was dissolved in 1 ml of deionized water. The water/oil emulsion was prepared by homogenization at 35,000 rpm for 1 minute and also followed by probe sonication for 5 minutes with a 30 second pause after each minute. This primary emulsion was added to 10 ml of a 1% w/v polyvinyl alcohol solution and homogenized using the same method as described for primary emulsion in order to obtain the final water/oil/water (w/o/w) emulsion. The emulsion was kept under stirring conditions for 6 hours to remove dichloromethane and the formulation was lyophilized and the dried particles were stored in glass vials at −20° C. for future use. BSA nanoparticles were prepared by a coacervation/nanoprecipitation method followed by lyophilization. Briefly, 270 mg of BSA and 27 mg of OT were dissolved in 10 ml of 10 mM NaCl at pH 9.3. Acetone was added drop wise at a speed of 1 ml/minute to the aqueous solution at a ratio of 2:1 with stirring at 800 rpm. After desolvation, the nanoparticle suspension was cross-linked for 30 minutes with 25% (w/v) glutaraldehyde in water (200 μL of glutaraldehyde for 1000 mg of BSA). Excess of glutaraldehyde was neutralized with sodium bisulphite. Trehalose (2% w/v) was added as a cryoprotectant and the nanoparticle suspension was lyophilized as above.

### Example 4: Tf and RVG Conjugation to Peptide-Loaded Nanoparticles

Previous studies have used Tf and RVG as brain targeting ligands [14, 18, 40] using other formulation technologies and to carry therapeutics other than neuropeptides into the brain. It was attempted to adopt these approaches for nanoparticle formulations of neuropeptides. Tf and RVG were conjugated to PLGA and BSA nanoparticles in two steps. The first step required activation of the carboxylic acid terminal groups using 1-ethyl-3-(3-dimethylaminopropyl) carbodimide (EDC) and NHS (98%) followed by conjugating Tf or RVG in the nanoparticles. Briefly, 1 ml (1 mg/ml) of EDC and NHS (1 ml, 1 mg/ml) was added to 10 ml of nanoparticle suspension containing 40 mg of OT-loaded PLGA or BSA nanoparticles. Carboxylic acid groups at the periphery were converted to amine-reactive esters by stirring the PLGA with EDC and sulfo-NHS reaction mixture at room temperature for 4 h. The activated NPs were then dispersed in 1 ml of PBS, and Tf (1 ml, 1 mg/ml) or RVG (1 ml, 1 mg/ml) was added drop-wise to the mixture. The mixture was stirred at room temperature for 2 h and incubated at 4° C. overnight or 12 hrs. Finally, the samples were washed and lyophilized using a Labconco freeze dryer (Labconco corporation, Missouri, USA). The 4 lead formulations were prepared as listed in Table 1

TABLE 1
Four lead formulations

| Number | Method of preparation | Formulation |
|---|---|---|
| 1 | Sonication/Solvent evaporation | TF conjugated to |
| | | PLGA nanoparticles |
| 2 | Sonication/Solvent evaporation | RVG conjugated to |
| | | PLGA nanoparticles |
| 3 | Coacervation/nanoprecipitation | TF conjugated to |
| | | BSA nanoparticles |
| 4 | Coacervation/nanoprecipitation | RVG conjugated to |
| | | BSA nanoparticles |

Tf = Transferrin;
RVG = Rabies Virus Glycoprotein;
PLGA = poly(lactic-co-glycolic acid);
BSA = Bovine serum albumin

### Example 5: Particle Size, Zeta Potential and Surface Morphology

To determine the size and zeta potential of the nanoparticle formulations, a 50 μL aliquot of the nanoparticle suspension was diluted with 1.5 mL distilled water. Malvern Zetasizer (Malvern Instruments Inc., Massachusetts, USA) was used to measure the particle size and zeta potential of the nanoparticles utilizing the principle of dynamic light scattering. The results reported are average values from triplicate runs of three independent experiments for each sample. To visualize surface morphology, microparticles were mounted onto metal stubs using double-sided adhesive tape. After being vacuum coated with a thin layer)(100-150A° of gold, the microspheres were observed under 20 kV using a scanning electron microscope (Phenom World Pure Scanning electron microscope, AZ, USA) to examine the morphology of the particles.

### Example 6: Peptide Encapsulation Efficiency

To determine the encapsulation efficiency, a known amount of each nanoparticle suspension was centrifuged at a speed of 35,000 rpm. The supernatant was obtained and the amount of peptide in the supernatant was determined by an OT-specific enzyme linked immunosorbent assay (ELISA) (Oxytocin ELISA kit, Catalogue no: ADI-901-153A-0001, Lot no: 12041404E, Enzo life Sciences, NY 11735). This amount was subtracted from the total amount of peptide used for the formulation to determine the peptide encapsulation efficiency inside the nanoparticles.

### Example 7: Fourier Transform Infrared Spectroscopy (FT-IR) to Determine Conjugation Efficiency

The adsorption of Tf and RVG on the surface of PLGA and BSA nanoparticles was investigated using FT-IR spectroscopy in order to determine the conjugation efficiency. The FT-IR spectra were recorded using IRAffinity—1S Spectrometer (Shimadzu Corporation, Japan). FTIR spectra of transferrin and transferrin (Tf) and RVG coated PLGA and BSA nanoparticles were obtained in the range of 4000 to 600 cm⁻¹. Spectral output was recorded by the transmittance as a function of wave number.

### Example 8: Immunogenicity

The potential of the formulations to induce an immunogenic reaction was assessed by measuring the amount of nitrite released by dendritic cells (DC) in presence of nanoparticulate formulations. Murine dendritic cells (DC 2.4, ATCC) were grown and maintained in DMEM with glucose, L-glutamine, and 10% FBS. DC cells were passaged at 70% confluence. For the assay, cells were plated in a 96 well-plate at 15,000 cells/well. The treatments consisted of untreated cells (negative control), the 4T1 immunogenic breast cancer vaccine (positive control), blank particles, and peptide particles. The cells were pulsed with 300 μg blank nanoparticles (1 mg/ml stock suspension) or peptide nanoparticles (1 mg/ml stock suspension) using incomplete DMEM in a 96-well plate for 16 hrs (n=3). After 16 hours, the supernatant was collected and analyzed for nitric oxide concentration using the Greiss chemical method. The Greiss reagent was prepared by mixing equal volumes of 1% sulfanilamide and 0.1% N-(1-napthylethyldiamine) solutions. One hundred microliters of the supernatant was transferred to a 96 well plate, to which 100 μl of Greiss reagent was added. The plate was incubated for 10 minutes and read at 540 nm to assess the color change using a microplate reader (Synergy H1; BIO-TEK Instruments, Winooski, Vt.). The concentration of nitrite was calculated for each well of cells using the standard curve of NaNO2 (1 mM stock concentration in distilled water further diluted to the highest standard at 100 μM followed by serial dilutions to 1.56 μM).

### Example 9: Cytotoxicity

The cytotoxicity of blank and peptide loaded nanoparticles was assessed by the MTT (3-(4,5-Dimethylthiazol-2-yl)-2,5-diphenyltetrazolium bromide) assay. Cell suspension equivalent to 10,000 cells/well was plated in a 96-well cell culture plate, and complete DMEM medium was added to obtain a volume of 100 μL per well. Various concentrations of the nanoparticles ranging from 0.5 μg/mL to 1000 μg/mL were added to the plates in triplicates and incubated for specified time points (24 and 48 hours). Cells alone (untreated) and benzalkonium chloride were used as negative and positive controls, respectively. 10 μL of MTT Reagent was added to each well, including controls followed by incubation at 37° C. for 2 to 4 hours. When the purple precipitate was clearly visible 100 μL of detergent reagent was added to all wells, including controls. Absorbance in each well was measured at 570 nm in a microtiter plate reader. The formation of a colored product quantified by measuring absorbance can be correlated to the percentage of living cells.

### Example 10: In Vitro Release

The release of OT from nanoparticles was studied in phosphate buffered saline (PBS, pH 7.4) and in 10% mice serum collected from Swiss-Webster mice by submandibular bleeding [41]. Briefly, 10 mg of nanoparticles were suspended in 1 mL of PBS or 10% mice serum and kept for continuous shaking (100 rpm). Two different temperatures were used, 4° C. and 37° C. At each time point, the tube was centrifuged at 35,000 rpm for 15 min, a sample of the supernatant was removed, after which the removed sample volume was replenished with media. The supernatant (n=3) was analyzed for the amount of peptide (OT) released using an OT ELISA as mentioned earlier.

### Example 11: Drugs/Chemicals

Poly(D,L-lactide-co-glycolide) lactide:glycolide (50:50), Polyvinyl Alcohol, Dichloromethane and Indocyanine green (ICG) were purchased from Sigma Aldrich (Milwaukee, Wis., USA). Bovine serum albumin (Fraction V), glutaraldehyde (25% in water) and acetone were purchased from Fischer Scientific (Pittsburgh, Pa., USA). All the chemicals were of analytical grade and used as is without further modification. OT GenScript USA, Piscataway, N.J.) was dissolved in a mixture of 10% Tween-80 and saline and lightly vortexed for suspension. Nanoparticles equivalent to 50 μg of oxytocin was suspended in 50 μl of vehicle for each intranasal administration. This dose was based on preliminary experiments.

### Example 12: Preparation of Nanoparticles

Bovine serum albumin (BSA) nanoparticles were prepared by a coacervation/nanoprecipitation method followed by lyophilization. Briefly, 270 mg of BSA and 27 mg of OT were dissolved in 10 ml of 10 mM NaCl at pH 9.3. Acetone was added drop wise at a speed of 1 ml/minute to the aqueous solution at a ratio of 2:1 with stirring at 800 rpm. After desolvation, the nanoparticle suspension was cross-linked for 30 minutes with 25% (w/v) glutaraldehyde in water (200 μL of glutaraldehyde for 1000 mg of BSA). Excess of glutaraldehyde was neutralized with sodium bisulphite. Trehalose (2% w/v) was added as a cryoprotectant and the nanoparticle suspension was lyophilized using Labconco freeze dryer (Labconco corporation, Missouri, USA).

### Example 13: Tf and RVG Conjugation to Peptide-Loaded Nanoparticles

Previous studies have used Tf and RVG as brain targeting ligands (Broadwell et al. 1996; Gan and Feng 2010; Kim et al. 2013; Liu et al. 2016; Pardridge 2014; Qian et al. 2002; Ulbrich et al. 2009) using other formulation technologies and to carry therapeutics other than neuropeptides into the brain. It was attempted to adopt their approaches for nanoparticle formulations of neuropeptides. Tf and RVG were conjugated to BSA nanoparticles in two steps. The first step required activation of the carboxylic acid terminal groups using 1-ethyl-3-(3-dimethylaminopropyl)carbodiimide (EDC) and NHS (98%) followed by conjugating Tf or RVG in the nanoparticles. Briefly, 1 ml (1 mg/ml) of EDC and NHS (1 ml, 1 mg/ml) was added to 10 ml of nanoparticle suspension containing 40 mg of OT-loaded BSA nanoparticles. The nanoparticles were dispersed in 1 ml of PBS, and Tf (1 ml, 1 mg/ml) or RVG (1 ml, 1 mg/ml) was added drop-wise to the mixture. The mixture was stirred at room temperature for 2 h and incubated at 4° C. overnight or 12 hrs. The samples were washed and lyophilized.

### Example 14: BEND3 Cell Culture

To generate a cellular model of the BBB, BEND3 cells were obtained from American Type Culture Collection (ATCC: 2299). BEND3 cells are endothelial cells that form tight barriers through the formation of transcellular tight junctions (Montesano et al. 1990; Sikorski et al. 1993; Williams et al. 1988), and these barriers mimic the barrier properties of the BBB (He et al. 2010; Watanabe et al. 2013). Their endothelial nature has been confirmed by the expression of von Willebrand factor and the uptake of fluorescently labeled low density lipoprotein. BEND3 cells were cultured in Dulbecco's Modified Eagle Medium (DMEM) supplemented with 15% FBS and 1% (100 U/ml Penicillin and 100 μg/ml Streptomycin). The cells were quickly thawed and centrifuged at 250×g for 8 minutes in 5 ml of complete media. The cell pellets were resuspended in 1 ml of complete media and plated on T-25 flask. Cells were maintained at 5% CO2, 37° C., and 80% humidity in a cell-culture incubator. At confluence, the cells were washed twice with 2.5 ml DPBS, trypsinized with TrypLE and centrifuged at 250×g for 8 minutes at 4° C. The cells were then resuspended in 1 ml of complete media and plated onto T-75 flask at appropriate densities for continuous passaging of the cells.

To characterize the membrane properties of the artificial BBB formed by these cells, the cells were seeded and cultured on permeable transwells using procedures optimized following the literature (Wuest et al. 2013). Confluent cells from T-75 flasks the cells were washed twice with 4 ml DPBS, trypsinized with TrypLE and centrifuged at 250×g for 8 minutes at 4° C. The cells were then resuspended in 1 ml of complete media. 150 of cell suspension was diluted with 150 of 0.4 trypan blue solution and a cell count was taken using a Countess II automated cell counting machine (MAN 0014293, Life Technologies, CA). The cells were diluted and seeded onto Costar permeable 12-well collagen coated membrane transwells with a 3 μm pore size (Corning; Corning, N.Y.) a density of 4×106 cells/insert in 0.5 ml of complete media. 1.5 ml of complete media was put on the basolateral side of the transwell while 0.5 ml of cell suspension was put into the apical side of the transwell insert. The media on both apical and basolateral sides of the transwell was changed every other day with complete media. All cells used for this work were below passage 33.

### Example 15: Transepithelial Electrical Resistance (TEER)

TEER measurements are a precise and well accepted method for assessing the barrier function of in vitro membranes prior to drug or formulation transport or barrier integrity studies (Li et al. 2004; Srinivasan et al. 2015). Following plating of the BEND3 cell on the transwells, the confluence of the cellular monolayer was manually monitored daily using microscopy. Furthermore, the resistance of the cellular monolayer was manually monitored daily using a Millicell-ERS-1 Epithelial Volt/Ohm Meter (EVOM Meter: MERS00001; MilliporeSigma; Burlington, Mass.). Briefly, the electrodes are sterilized with 70% ethanol and equilibrated in complete growth media. The resistance of the blank membrane was recorded as well as that of the cells, and the resistance of the blank membrane was subtracted from the resistance of the cell barrier. During resistance measurements, the electrodes are positioned such that the silver-silver chloride pellet neither touches the membrane of the insert nor is exposed to air. Actual voltage readings are obtained by subtracting blank resistance values. The resistance of cells was measured daily using the EVOM meter until resistance ranging from 50 to 100 ohms cm² or more was obtained. This typically occurred between 6 to 8 days following plating.

### Example 16: BBB Transport

To study the transport of nanoparticle formulations across the BEND3 BBB model barrier, a complete Ussing chamber system with a multichannel voltage-current clamp and the EasyMount Ussing chambers (Model VCC MC8; P2300; Physiologic Instruments Inc.; San Diego, Calif.) was used. The combination of the Ussing chambers and multichannel voltage-current clamp provided an apparatus for determining accurate nanoparticle transport and precise real-time measurements of the BEND3 membrane barrier function and bioelectric properties—TEER, short circuit current Isc, and potential difference PD. This system was used for final experiments once the cultures reached appropriate resistance as verified by EVOM measurements. To subtract the background resistance, a blank cell-culture insert was placed onto the slider and used to compensate for any fluid or blank membrane resistance by dialing off any reading to zero. Voltage differences between the electrodes were also offset. The blank inserts were then replaced with inserts containing cells and kept a temperature of 37° C. throughout the duration of the experiment. The cells were allowed to equilibrate in the Us sing chamber for 15 minutes before start of the experiment. Before the start of the experiment, all cell barrier preparations were evaluated for resistance properties and current flow using the acquire and analyze software. The resistance values of the cells during the experiment were obtained using the same software. For each experimental set up, 3 chambers contained cells while one was a blank insert.

For visual confirmation of monolayer formation, a solution (0.4%) of the visible dye trypan blue was used. This dye was chosen because its molecular weight (986.41 g/mol) is comparable to the molecular weight of OT (1006 g/mol). 100 μl of dye solution was put in the left side of each chamber and sampled from the right compartment at times 1, 2, 3 and 4 hours following dye administration. The intensity of dye at the various time points were quantified using a BioTek HT synergy spectrophotometer at 540 nm (Model S1AFR BioTek; Winooski, Vt.). To detect transport of the nanoparticles across the BBB, fluorescein isothiocyanate (FITC) was encapsulated in BSA nanoparticles and used to characterize transport of Tf-coated nanoparticles across the formed monolayer. 200 μl of nanoparticles solution of concentration 134 ug/ml solution was put in the left side of each chamber and sampled from the right compartment at times 1, 2, 3 and 4 hours. The fluorescent intensity of FITC at the various time points was quantified using Biotek fluorescent spectrophotometer at an excitation wavelength of 485 nm and an emission wavelength of 528 nm. The experiment was conducted in a dark environment to prevent fluorescent photobleaching of FITC.

### Example 17: Animals

Male Swiss-Webster mice (CFW; Charles River Laboratories, Inc.; Wilmington, Mass.) weighing between 25 and 40 g served as the subjects of the in vivo experiments. The mice were housed in groups of 4 in a temperature and humidity controlled room. Animals had access to food (Laboratory Rodent Diet) and water ad libitum. Mice were housed in rooms maintained in a 12-hour light/dark cycle. The lights were turned off at 6 pm every evening, and turned back on at 6 am every morning. All animals employed in this study were treated according to protocols evaluated and approved by Institutional Animal Care and Use Committee of Mercer University.

### Example 18: Bioimaging

On experimental days, animals were administered ICG-loaded nanoparticles (6.7 ug of dye equivalent) intranasally, anaesthetized with isoflurane, and imaged using a LI-COR Odyssey Bioimager equipped with a MousePOD for in vivo imaging (LI-COR Biosciences; Lincoln, Nebr.). All mice were imaged at times 0.25, 1.5, 3, 6 and 24 hours. During imaging, animals were kept under anesthesia with isoflurane. The integrated intensity of ICG dye in the brain at 800 nm was quantified by the bioimager. For both Tf and RVG nanoparticles, more than 90% of the dye was cleared from the brain in 24 hours, most likely due to degradation of the dye itself rather than clearance of the nanoparticles from the brain. As the peak intensity of ICG dye in the brain was at approximately 2 hours for both Tf and RVG nanoparticles, the 2 hour time point for the subsequent in vivo cerebrospinal fluid (CSF) analysis and acute social behavior experiments were used.

### Example 19: Collection of CSF and Plasma

Borosilicate glass capillary with filament (BD 100-75-10, Sutter Instrument Inc) was pulled in the middle over a Bunsen burner. The tip of the flamed glass capillary tubes was trimmed with scissors so that the tapered tip has an inner diameter of approximately 0.5 mm.

On sample collection days, animals were administered a 50 μl solution of OT alone or encapsulated in nanoparticles (containing 50 ug of OT) intranasally and placed in separate cages for 120 minutes.

CSF samples were collected from the cisterna magna as previously published by (Liu and Duff 2008) with little modifications. Briefly, the mice were anaesthetized with isoflurane and maintained under anesthesia at a flow rate of 1.5 ml/min during the surgical process. The mice were then placed prone in the stereotaxic instrument in direct contact with a heating pad with their heads firmly secured. The skin on the head and neck was shaved and the surgical site wiped with 10% povidone iodine followed by 70% ethanol. An incision was made and the muscles under the skin separated by blunt dissection under a dissecting microscope until the dura of the cisterna magna were exposed (a glistening white reverse triangle). The mice were then repositioned so the head formed a near 135° angle to the body. The dura mater was blotted dry with sterile cotton swab and saline. A capillary tube was inserted into the cisterna magna through the dura mater to allow CSF to flow into the glass capillary tube. The CSF was transferred into a pre-marked 0.6 ml Eppendorf, immediately placed on ice, and transferred into a −20° C. freezer. Any sample contaminated with blood (pink tinge) was discarded.

Immediately after CSF collection, blood samples were collected via the facial vein for each mouse into K2EDTA tubes (BD Microtainer 365974) and immediately placed on ice. The mouse was immediately euthanized by carbon dioxide asphyxiation. Blood samples were spun at 4200 rpm for 10 minutes at 4° C. after which the plasma fraction was collected and stored at −20° C. until analysis. Samples were not stored for more than 10 days.

### Example 20: Enzyme Linked Immunosorbent Assay (ELISA)

All collected CSF and Plasma samples were treated identically and assayed at the same time with the same batch of reagents. All samples were tested using commercially available oxytocin ELISA kit (ADI-900-153A, Enzo Life Sciences, NY). The assay was performed according to the protocol provided by the manufacturer. Briefly, seven standards and samples were diluted in assay buffer and put in appropriate wells. To ensure ELISA kit was working appropriately, quality control metrics such as the total activity (TA), nonspecific binding (NSB), maximum binding (BO) and blank wells were included in the assay as recommended in the assay protocol. After addition of yellow antibody to all wells except for blank, TA and NSB wells, the plate was tapped gently, sealed, and incubated at 4° C. for 20 hours. After three washes, blue conjugate was added to TA wells and pNpp substrate solution was immediately added to all wells and incubated at room temperature without shaking for 1 hour after which stop solution was added. The optical density was read immediately at 405 nm with correction at 570 nm using BioTek HT synergy spectrophotometer at 540 nm (Model S1AFR BioTek; Winooski, Vt.). The mean optical density of the blank wells was subtracted from all readings.

### Example 21: Social Behavior

Social-behavior experiments consisted of dyadic social interactions between familiar mice (mice housed in the same home cage). All experiments were conducted in boxes (33×25×9 cm) large enough to provide for social separation, with ambient lighting dim enough to allow interactions but bright to enable scoring of the videos. The bedding used for all experiments was from the home cages of the animals to prevent novelty-induced hyper-excitability. Experiments were conducted with the room kept at a minimal noise level. All videos were scored by a trained observer. On experimental days, animals were administered a 50 μl suspension of OT alone or encapsulated in nanoparticles (containing 50 μg of OT) intranasally and placed in separate cages for 120 minutes (n=4 to 6 pairs per group). The mice were then placed in the testing box, allowed to acclimatize in the box for 5 minutes and video recorded for 10 minutes. Behavior was rated on a 6-point scale, composed of: adjacent lying, anogenital sniffing, general investigation, body crossing, aggression, and no social interaction, as has been done previously (Curry et al. 2018).

### Example 22: Data Analysis

All graphical data presentations and analyses were completed using GraphPad Prism (GraphPad Software Inc., La Jolla, Calif., USA). The cell culture data were analyzed by two-way analysis of variance (ANOVA). Bonferroni post-hoc analyses test was utilized to maintain a 95% confidence interval. The in vivo bioimaging data were analyzed by one-way ANOVA followed by Dunnett's post-hoc test. CSF concentrations of OT were assessed by unpaired t-test. The in vivo social behavior data were analyzed by one-way ANOVA followed by Tukey's post-hoc test. Post-hoc tested corrected for multiple planned comparisons to maintain the probability of making a type 1 error at 5%.

### Example 23: Particle Size, Zeta Potential and Surface Morphology

In general, the smaller the size of the nanoparticles, the more amenable they are for active transport across the BBB [42-45]. The mean diameter of PLGA nanoparticles measured by Malvern zeta-sizer ranged between 197.7 and 278.3 nm. PLGA-based particles were negatively charged ranging from −11.9 to −19.6 mV. The mean diameter of the BSA nanoparticles ranged from 100.1 to 197 nm in diameter. All the BSA nanoparticles were negatively charged ranging from −15.4 to −22.8 mV. The lowest particle size (approx. 100 nm) was obtained using a BSA formulation. Size distribution of RVG-conjugated OT-loaded BSA nanoparticles showed two peaks where intensity of the main peak was 99.24%. This may happen due to aggregation of some nanoparticles. As more than 99% particles were within a very narrow distribution range, it can be said that very few particles had aggregated. For Tf-conjugated OT-loaded BSA nanoparticles, the intensity of the main peak was 92.6%. For RVG- and TF-conjugated OT-loaded PLGA nanoparticles there was only one peak for each type of particles and the intensities of the peaks were 99.9% and 100% respectively.

FIGS. 1A-1C show representative data from the Zetasizer as well as the scanning electron microscope. As can be seen, the particle formulations exhibited an even spherical shape. Table 2 summarizes the sizes and zeta potentials of the various formulations that were assessed.

TABLE 2
Size distribution and Zeta potentials of different PLGA and
BSA nanoparticle formulations of oxytocin.

| | | | | Characteristics | |
|---|---|---|---|---|---|
| Formulation | | | | | Zeta |
| Base | | Targeting | | Size | Potential |
| Material | Loading | Ligand | Preparation Method | (nm) | (mV) |
| PLGA | Blank | Tf | Homogenization | 240 | −20.10 |
| PLGA | OT | Tf | Homogenization | 278.3 | −16.4 |
| PLGA | Blank | Tf | Sonication | 205.2 | −16.3 |
| PLGA | OT | Tf | Sonication | 191.7 | −19.6 |
| PLGA | Blank | RVG | Sonication | 203.44 | −16.5 |
| PLGA | OT | RVG | Sonication | 201.2 | −17.4 |
| BSA | Blank | Tf | Coacervation/ | 192.9 | −19.6 |
| | | | nanoprecipitation | | |
| BSA | OT | Tf | Coacervation/ | 196.3 | −21.1 |
| | | | nanoprecipitation | | |
| BSA | Blank | RVG | Coacervation/ | 137 | −20.5 |
| | | | nanoprecipitation | | |
| BSA | OT | RVG | Coacervation/ | 100.1 | −16.0 |
| | | | nanoprecipitation | | |

Tf = Transferrin;
RVG = Rabies Virus Glycoprotein;
PLGA = poly(lactic-co-glycolic acid);
BSA = Bovine serum albumin;
OT = Oxytocin

### Example 24: Optimization of Formulation by Full Factorial Design

Parameters affecting the particle size are rank ordered by analysis of the formulation design with JMP® (SAS) software. Significant parameters (p<0.05) affecting particle size (in the order of importance) are interaction of BSA concentration and acetone: water ratio (p<0.0005) pH of the aqueous solution (p<0.0006), interaction of pH and acetone: water ratio (p<0.0013), acetone: water ratio (p<0.0053). The optimization is listed in Table 3.

TABLE 3
Parameters that significantly affect the particle size as
revealed by full factorial design of formulation variables
for bovine serum albumin (BSA) nanoparticles.

| Parameters affecting particle size | Significance |
|---|---|
| Interaction of BSA concentration and acetone:water ratio | p < 0.001 |
| pH of the aqueous solution | p < 0.001 |
| Interaction of pH and acetone:water ratio | p < 0.001 |
| Acetone:water ratio | p = 0.005 |

### Example 23

Transferrin (Tf) and Rabies Virus Glycoprotein (RVG) Conjugation to Peptide Loaded Nanoparticles

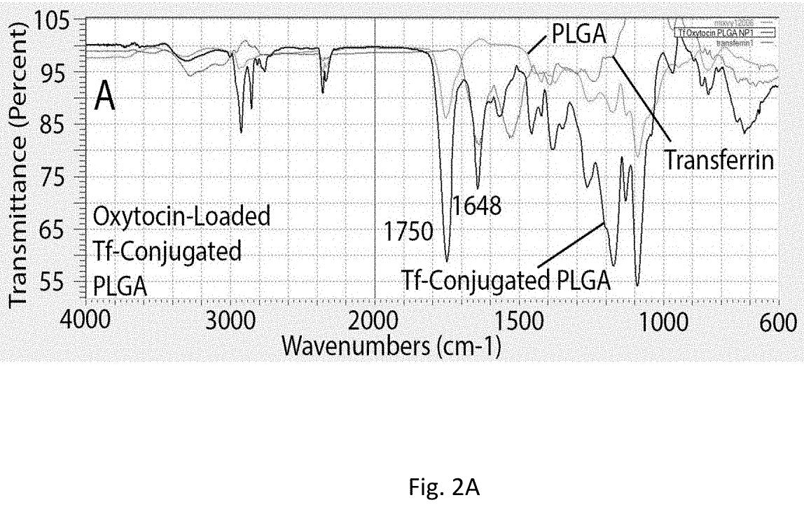

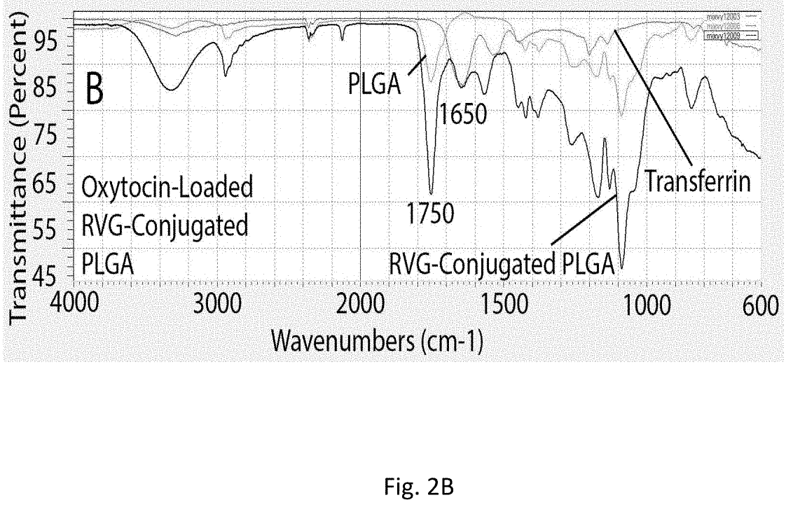

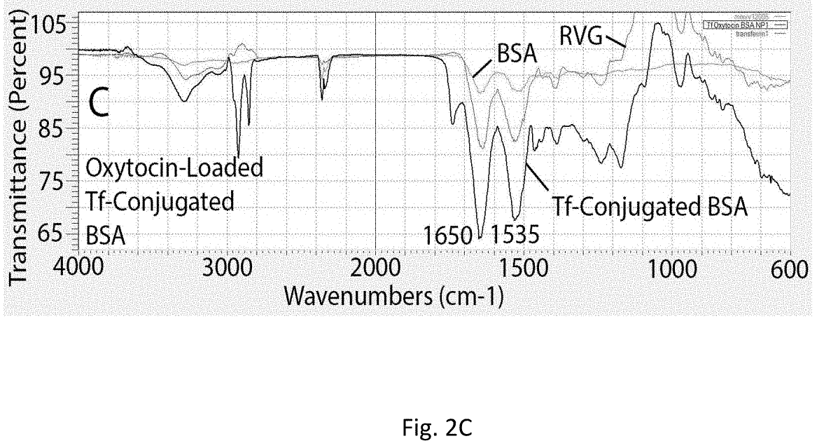

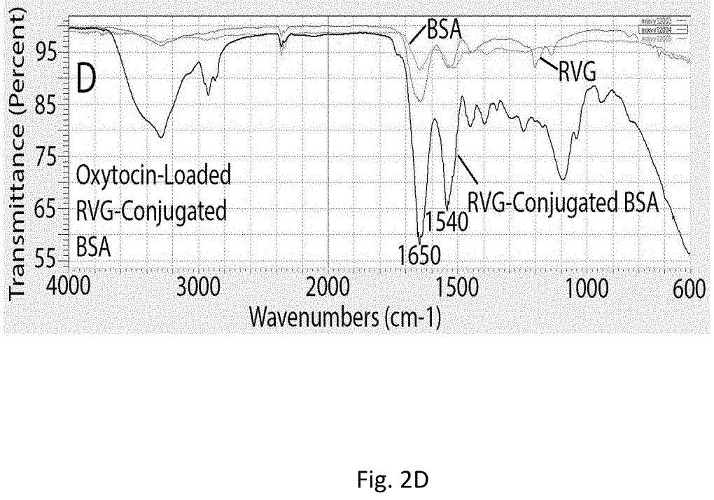

FIGS. 2A-2C show the FT-1R spectra of Tf- and RVG-unconjugated and conjugated PLGA nanoparticles. FIG. 2 (A) shows two main peaks at 1640 cm-1 and 1535 cm-1, respectively the two peaks of amide I and amide II vibrations of Tf and RVG. 2 (B) shows FT-IR spectrum of unmodified PLGA nanoparticles having characteristic bands of carbonyl C═O stretching (1750 cm-1) and the ester C—O stretching (1000-1300 cm-1). FIG. 2(C) shows the FT-IR spectrum of PLGA nanoparticles conjugated with Tf showing the amide bands assigned to the protein. The peak of the amide I band in the spectrum of PLGA-Tf nanoparticles occurred at 1648 cm-1. The bands of the characteristic functional groups of PLGA nanoparticles are present (1750 cm-1 for C═O stretching and 1000-1300 cm-1 for the ester C—O stretching), with small shifts when compared to the spectrum of unconjugated nanoparticles. The presence of the amide bond confirms the presence of Tf on the Tf-coated PLGA nanoparticles.

FIGS. 2 (C and D) show the FT-IR spectra of RVG and Tf conjugated BSA nanoparticles. Both spectra shows two main peaks at 1640 cm-1 and 1535 cm-1, respectively the two peaks of amide I and amide II vibrations of Tf and RVG. Same two peaks are visible for BSA also. Both RVG- and Tf-conjugated BSA nanoparticles shows intensified peaks for amide I and amide II vibrations indicating conjugation of RVG and Tf to BSA.

### Example 25: Encapsulation Efficiency

The encapsulation efficiency was determined before the conjugation of the nanoparticles to Tf and RVG. The amount of peptide in the supernatant was subtracted from the total amount of peptide used for the formulation to determine the peptide encapsulation efficiency inside the nanoparticles. The oxytocin peptide content of the PLGA nanoparticles was 81±2.5% (w/w) and for BSA nanoparticles it was 75±2.5% (w/w).

### Example 26: Immunogenicity Determination

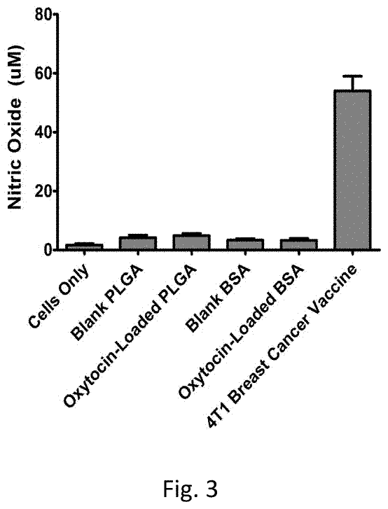

Nitric oxide release is an important indicator of innate immune response [46,47]. This can be used to determine the potential of nanoparticle formulations to induce immunogenic reactions. FIG. 3 shows the effects of each formulation of oxytocin on nitric oxide release. None of the blank or OT-loaded nanoparticles induced significant nitric oxide release demonstrating non immunogenicity which is desirable when compared to 4T1 murine tumor antigen loaded microparticles, which resulted in a large NO release and high immunogenicity as the control immunogenicity response. All experiments were conducted in triplicate.

### Example 27: Cell Cytotoxicity

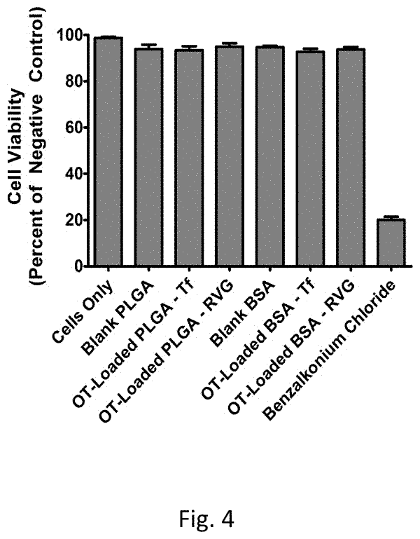

The cytotoxic effects of the four lead nanoparticle formulations (as mentioned in Table 1) at given concentrations are depicted in FIG. 4. No formulation was cytotoxic within the tested concentration range (0.0625-1 mg/mL) after 48 hours. The percent cell viability is expressed relative to negative controls of cells treated with DMEM medium only. Positive control of benzalkonium chloride was cytotoxic, as it yielded 21.44% and 20.089% cell viability after 24 and 48 hours, respectively. All experiments were done in triplicate.

### Example 28: In Vitro Release

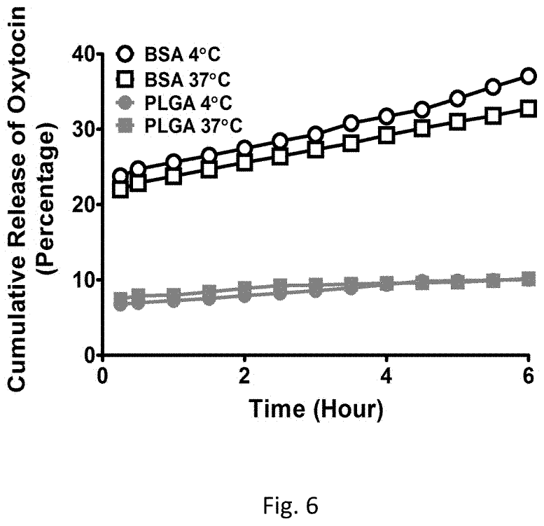

The release of OT from PLGA nanoparticles in PBS was sustained over a period of 21 days (FIG. 5). OT was released from PLGA nanoparticles in a sustained fashion and 50% of peptide was released over a period of 10.8 days (FIG. 5). The release of OT from BSA nanoparticles in PBS was sustained over a period of 18 days with 50% of OT released over a period of 9.8 days (FIG. 5). In 10% serum at pH 7.4 OT is released from BSA nanoparticles in sustained fashion, with initial burst release of around 20% and 50% of OT released over a period of 3 days at both 4° C. and 37° C. To get a clearer view of the burst release, a release study for 6 hours with sampling interval of 30 minutes was conducted. At the end of 6 hours 37.07% and 32.8% OT was released from BSA nanoparticles at 4° C. and 37° C. temperature, respectively (FIG. 6). The release of OT from PLGA nanoparticles was very slow with only 5% release at the end of 6 hours at both 4° C. and 37° C. (FIG. 6). These data show that BSA nanoparticles have a faster initial release profile than PLGA particles, and that temperature does not have a significant effect on the release profile over the first 6 hours. The goal was to find a release profile that provides an initial burst release of the peptide to induce rapid symptom control followed by a sustained release for prolonged therapeutic effects. FIG. 7 demonstrates that the RVG-conjugated BSA nanoparticles have a mixed burst release/sustained release profile, with approximately 35% release within the first 6 hours, but 50% release not occurring until approximately 3 days in solution.

### Example 29: TEER Trypan Blue

The integrity of the artificial BBB barrier was assessed using trypan blue transport. The resistance of cellular barrier (BEND3 cells) at first sampling point, t=60 min was Rt 123.7+54 Ω·cm2 n=3 and at t=240 min was Rt 117.2+42.5 Ω·cm2; n=3. Resistance without a cellular barrier, t=60 min was Rt 2.89+1.2 Ω·cm2 n=3 and at t=240 min was Rt 4.2+2.7 Ω·cm2 n=15.

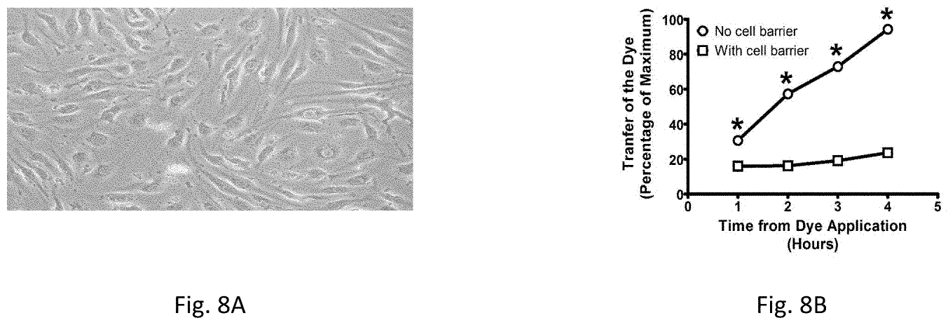

The transport of trypan blue across cell barrier was (FIGS. 8A and 8B) assessed by determining the main effect of the presence or absence of the cell barrier and the main effect of time by two-way ANOVA. There was a significant main effect of the presence of the cell barrier (F1,16=6762; p<0.0001). There was a significant main effect of time (F3,16=952.1; p<0.0001). There was also a significant interaction (F3,16=520.2; p<0.0001). Post-hoc analysis revealed significant (t=13.02, 35.38, 48.93, and 67.14) reductions of trypan blue passage across the cell barrier at 1, 2, 3 and 4 hours. There was significant reduction in the transport of trypan blue in the presence of the cell barrier at all time points (p<0.001 at all time points).

### Example 30: FITC

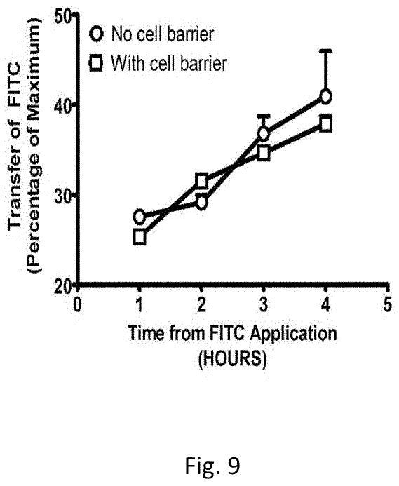

Transport of FITC in brain targeting nanoparticles across the artificial BBB barrier (FIG. 9) was assessed by determining the main effect of the presence or absence of the cell barrier and the main effect of time by two-way ANOVA. There was no significant main effect of the barrier (F1,16=0.79; p<0.3844). There was a significant main effect of time (F3,16=16.90; p<0.0001). There was no significant interaction between these factors (F3,16=0.7816; p<0.5214). Post-hoc analysis revealed (t=0.7860, 0.8593, 0.7654, 1.097) no significant differences due to the presence of the cellular barrier at any time point. There were no significant differences in transport of FITC in brain targeting nanoparticles due to the presence or absence of the cell barrier, indicating that the brain targeting particles rapidly surmount the barrier, most likely through active transport across this barrier. The resistance of the cellular barrier (BEND3 cells) at the first sampling point (t=60 min) was Rt 107.28+31.7 Ω·cm2 (n=5), and at the end of the experiment (t=240 min), it was Rt 127.22+28 Ω·cm2 (n=5). Without the cellular barrier, the resistance of the cellular barrier at the first sampling point (t=60 min) was Rt 5.03+1.8 Ω·cm2 (n=5), and at the end of the experiment (t=240 min), it was t=240 min Rt 10.8+2 Ω·cm2 (n=5).

### Example 31: Bioimaging

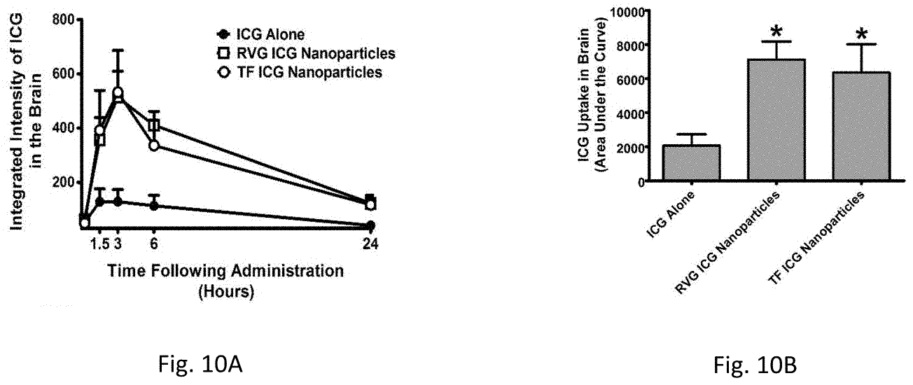

The time course of brain uptake of ICG alone or ICG in each of the two formulations was determined. The area under the curve (AUC) of the time course in each animal was determined and tabulated for statistical analyses. There was a significant main effect of treatment (F2,12=5.098; p<0.0250) on brain penetrance of ICG nanoparticles (FIGS. 10A and 10B). Post-hoc analysis by Dunnett's test revealed that OT in RVG-conjugated nanoparticles (q=2.960) at a concentration of 6.7 μg and OT in Tf-conjugated nanoparticles (q=2.518) at a concentration of 6.7 μg were significantly different from ICG solution group.

### Example 32: Plasma and CSF Analysis

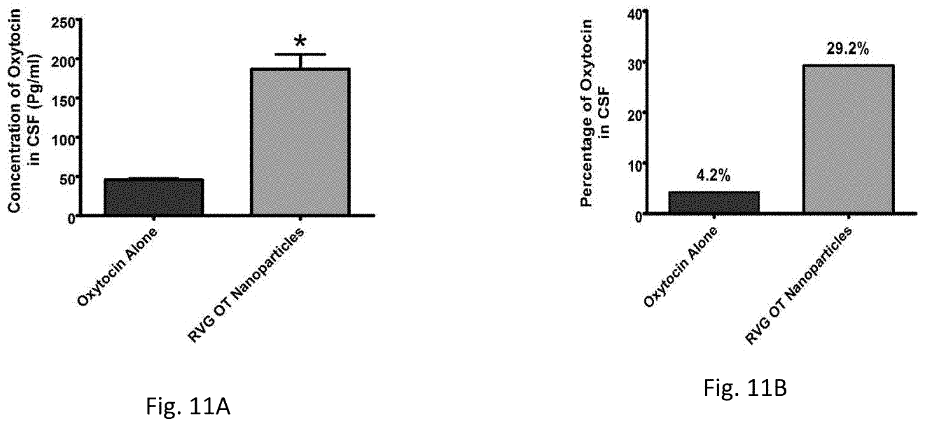

Consistent with the bioimaging data, 2 hours after intranasal administration, significantly higher levels of OT in CSF (t=7.501; p<0.05) when it was administered in RVG-conjugated nanoparticles compared to when it was administered alone (FIG. 11A) was observed. Furthermore, a decrease in plasma levels of OT following RVG-conjugated nanoparticles was observed, but this decrease did not reach statistical significance. The proportion of OT in CSF versus the total in CSF plus plasma was calculated, and a significantly higher proportion (t=10.48; p<0.01) of OT in CSF following intranasal administration in RVG-conjugated nanoparticles (22.9%) compared to when it was administered alone (4.2%) (FIG. 11B) was observed.

### Example 33: Social Behavior

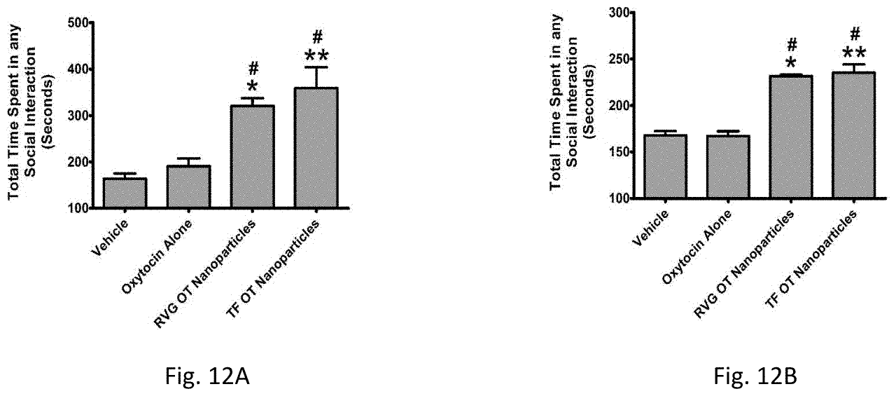

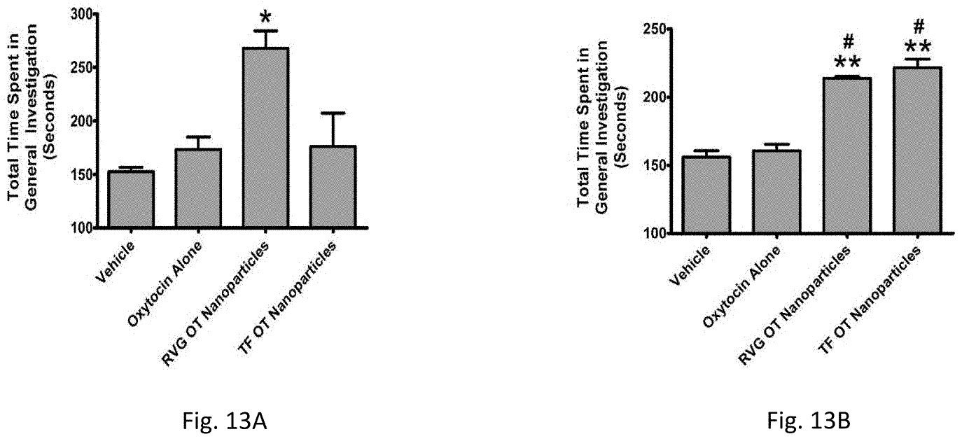

One-way ANOVA revealed a significant main effect of treatment (F3,32=8.897; p<0.0002) on murine social interactions (FIG. 12A). Post-hoc analysis by Tukey's test revealed that OT in RVG-conjugated nanoparticles (q=4.530) or Tf-conjugated nanoparticles (q=6.182) at a concentration of 50 μg was significantly different from vehicle. Post-hoc analysis also showed that OT in Tf-conjugated nanoparticles (q=5.317) was significantly different from 50 μg of OT alone. One-way ANOVA of social behavior 3 days after treatment revealed a significant main effect (F3,32=8.897; p<0.0001) of treatment on overall social interactions (FIG. 12B). Post-hoc analysis by Tukey's test revealed that OT in OT in RVG-conjugated nanoparticles (q=8.757) or Tf-conjugated nanoparticles (q=10.14) was significantly different from vehicle. Moreover, OT in RVG-conjugated nanoparticles (q=8.871) or Tf-conjugated nanoparticles (q=10.27) was significantly different from OT alone. General Investigation was been previously reported to be the dominant social parameter (Curry et al. 2018) when mice are given the highly prosocial and brain penetrant small molecule 3,4-methylenedioxymethamphetamine (MDMA) (Dumont et al. 2009), which is known release OT (Harris et al. 2002; Murnane et al. 2010; Murnane et al. 2012; Thompson et al. 2007). Consistent with those data, the dominant social behavior in the mice was general investigation both after acute administration of nanoparticle encapsulated OT and 3 days later. One-way ANOVA revealed a significant main effect (F3,32=4.336; p<0.0113) of treatment on general investigation following acute treatment (FIG. 13A). Post-hoc analysis Tukey's test revealed that OT in RVG-conjugated nanoparticles (q=4.460) was significantly different from vehicle. OT in RVG-conjugated nanoparticles (q=4.043) was significantly different from OT in Tf-conjugated nanoparticles. After 3 days, there was a significant main effect of treatment (F3,32=43.84; p<0.0006) on general investigation (FIG. 13B). Post-hoc analysis revealed that OT in RVG-conjugated nanoparticles (q=10.41) or Tf-conjugated nanoparticles (q=12.91) was significantly different from vehicle. Also, OT in RVG-conjugated nanoparticles (q=9.575) or Tf-conjugated nanoparticles (q=12.00) was significantly different from OT alone.

Although only a number of exemplary embodiments have been described in detail above, those skilled in the art will readily appreciate that many modifications are possible in the exemplary embodiments without materially departing from the novel teachings and advantages. Accordingly, all such modifications are intended to be included within the scope of this disclosure as defined in the following claims.

While the methods, equipment and systems have been described in connection with specific embodiments, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods, equipment and systems. These and other components are disclosed herein, and it is understood that when combinations, subsets, interactions, groups, etc., of these components are disclosed that while specific reference of each various individual and collective combinations and permutation of these may not be explicitly disclosed, each is specifically contemplated and described herein, for all methods, equipment and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

It should further be noted that all patents, applications, and publications referred to herein are incorporated by reference in their entirety.

### REFERENCE LIST

[1] Misra A, Ganesh S, Shahiwala A, Shah S P. Drug delivery to the central nervous system: a review. Journal of pharmacy & pharmaceutical sciences: a publication of the Canadian Society for Pharmaceutical Sciences, Societe canadienne des sciences pharmaceutiques. 2003; 6:252-73.
[2] Broderick D F, Schweitzer K J, Wszolek Z K. Vascular risk factors and dementia: how to move forward? Neurology. 2009; 73:1934-5.
[3] Herrmann N, Chau S A, Kircanski I, Lanctot K L. Current and emerging drug treatment options for Alzheimer's disease: a systematic review. Drugs. 2011; 71:2031-65.
[4] Strecker K, Schwarz J. Parkinson's disease: emerging pharmacotherapy. Expert opinion on emerging drugs. 2008; 13:573-91.
[5] Carson D S, Guastella A J, Taylor E R, McGregor I S. A brief history of oxytocin and its role in modulating psychostimulant effects. Journal of psychopharmacology (Oxford, England). 2013; 27:231-47.
[6] Donaldson Z R, Young L J. Oxytocin, vasopressin, and the neurogenetics of sociality. Science (New York, N.Y. 2008; 322:900-4.
[7] Engelmann M, Wotjak C T, Neumann I, Ludwig M, Landgraf R. Behavioral consequences of intracerebral vasopressin and oxytocin: focus on learning and memory. Neurosci Biobehav Rev. 1996; 20:341-58.
[8] Modi M E, Young L J. The oxytocin system in drug discovery for autism: animal models and novel therapeutic strategies. Horm Behay. 2012; 61:340-50.
[9] Nagahara A H, Merrill D A, Coppola G, Tsukada S, Schroeder B E, Shaked G M, et al. Neuroprotective effects of brain-derived neurotrophic factor in rodent and primate models of Alzheimer's disease. Nature medicine. 2009; 15:331-7.
[10] Dawbarn D, Allen S J. Neurotrophins and neurodegeneration. Neuropathology and applied neurobiology. 2003; 29:211-30.
[11] O'Brien R J, Wong P C. Amyloid precursor protein processing and Alzheimer's disease. Annu Rev Neurosci. 2011; 34:185-204.
[12] Opal S M, DePalo V A. Anti-inflammatory cytokines. Chest. 2000; 117:1162-72.
[13] Walker D, Lue L F. Anti-inflammatory and immune therapy for Alzheimer's disease: current status and future directions. Current neuropharmacology. 2007; 5:232-43.
[14] Blesch A, Tuszynski M H. Transient growth factor delivery sustains regenerated axons after spinal cord injury. J Neurosci. 2007; 27:10535-45.
[15] Sloane E, Ledeboer A, Seibert W, Coats B, van Strien M, Maier S F, et al. Anti-inflammatory cytokine gene therapy decreases sensory and motor dysfunction in experimental Multiple Sclerosis: MOG-EAE behavioral and anatomical symptom treatment with cytokine gene therapy. Brain, behavior, and immunity. 2009; 23:92-100.
[16] Soderquist R G, Mahoney M J. Central nervous system delivery of large molecules: challenges and new frontiers for intrathecally administered therapeutics. Expert opinion on drug delivery. 2010; 7:285-93.
[17] Pulicherla K K, Verma M K. Targeting therapeutics across the blood brain barrier (BBB), prerequisite towards thrombolytic therapy for cerebrovascular disorders—an overview and advancements. AAPS PharmSciTech. 2015; 16:223-33.
[18] Gan C W, Feng S S. Transferrin-conjugated nanoparticles of poly(lactide)-D-alpha-tocopheryl polyethylene glycol succinate diblock copolymer for targeted drug delivery across the blood-brain barrier. Biomaterials. 2010; 31:7748-57.
[19] Pardridge W M. Blood-brain barrier delivery. Drug discovery today. 2007; 12:54-61.
[20] Gatter K C, Brown G, Trowbridge I S, Woolston R E, Mason D Y. Transferrin receptors in human tissues: their distribution and possible clinical relevance. Journal of clinical pathology. 1983; 36:539-45.
[21] Pang Z, Gao H, Yu Y, Guo L, Chen J, Pan S, et al. Enhanced intracellular delivery and chemotherapy for glioma rats by transferrin-conjugated biodegradable polymersomes loaded with doxorubicin. Bioconjugate chemistry. 2011; 22:1171-80.
[22] Qian Z M, Li H, Sun H, Ho K. Targeted drug delivery via the transferrin receptor-mediated endocytosis pathway. Pharmacol Rev. 2002; 54:561-87.
[23] Ulbrich K, Hekmatara T, Herbert E, Kreuter J. Transferrin- and transferrin-receptor-antibody-modified nanoparticles enable drug delivery across the blood-brain barrier (BBB). European journal of pharmaceutics and biopharmaceutics: official journal of Arbeitsgemeinschaft fur Pharmazeutische Verfahrenstechnik eV. 2009; 71:251-6.
[24] Wiley D T, Webster P, Gale A, Davis M E. Transcytosis and brain uptake of transferrin-containing nanoparticles by tuning avidity to transferrin receptor. Proc Natl Acad Sci USA. 2013; 110:8662-7.
[25] Yan F, Wang Y, He S, Ku S, Gu W, Ye L. Transferrin-conjugated, fluorescein-loaded magnetic nanoparticles for targeted delivery across the blood-brain barrier. Journal of materials science Materials in medicine. 2013; 24:2371-9.
[26] Ying X, Wen H, Lu W L, Du J, Guo J, Tian W, et al. Dual-targeting daunorubicin liposomes improve the therapeutic efficacy of brain glioma in animals. Journal of controlled release: official journal of the Controlled Release Society. 2010; 141:183-92.
[27] Broadwell R D, Baker-Cairns B J, Friden P M, Oliver C, Villegas J C.

Transcytosis of protein through the mammalian cerebral epithelium and endothelium. III. Receptor-mediated transcytosis through the blood-brain barrier of blood-borne transferrin and antibody against the transferrin receptor. Experimental neurology. 1996; 142:47-65.
[28] Pardridge W M. Blood-brain barrier drug delivery of IgG fusion proteins with a transferrin receptor monoclonal antibody. Expert opinion on drug delivery. 2014:1-16.
[29] Li Y, Ogris M, Wagner E, Pelisek J, Ruffer M. Nanoparticles bearing polyethyleneglycol-coupled transferrin as gene carriers: preparation and in vitro evaluation. International journal of pharmaceutics. 2003; 259:93-101.
[30] Pan X, Guan J, Yoo J W, Epstein A J, Lee L J, Lee R J. Cationic lipid-coated magnetic nanoparticles associated with transferrin for gene delivery. International journal of pharmaceutics. 2008; 358:263-70.
[31] Sahoo S K, Labhasetwar V. Enhanced antiproliferative activity of transferrin-conjugated paclitaxel-loaded nanoparticles is mediated via sustained intracellular drug retention. Molecular pharmaceutics. 2005; 2:373-83.
[32] Zhang X, Koh C G, Yu B, Liu S, Piao L, Marcucci G, et al. Transferrin receptor targeted lipopolyplexes for delivery of antisense oligonucleotide g3139 in a murine k562 xenograft model. Pharmaceutical research. 2009; 26:1516-24.
[33] Liu Y, An S, Li J, Kuang Y, He X, Guo Y, et al. Brain-targeted co-delivery of therapeutic gene and peptide by multifunctional nanoparticles in Alzheimer's disease mice. Biomaterials. 2016; 80:33-45.
[34] Makadia H K, Siegel S J. Poly Lactic-co-Glycolic Acid (PLGA) as Biodegradable Controlled Drug Delivery Carrier. Polymers. 2011; 3:1377-97.
[35] Tong W, Wang L, D'Souza M J. Evaluation of PLGA microspheres as delivery system for antitumor agent-camptothecin. Drug development and industrial pharmacy. 2003; 29:745-56.
[36] Oettinger C W, D'Souza M J, Akhavein N, Peer G T, Taylor F B, Kinasewitz G T. Pro-inflammatory cytokine inhibition in the primate using microencapsulated antisense oligomers to N F-kappaB. J Microencapsul. 2007; 24:337-48.
[37] Kolluru L P, Rizvi S A, D'Souza M, D'Souza M J. Formulation development of albumin based theragnostic nanoparticles as a potential delivery system for tumor targeting. Journal of drug targeting. 2013; 21:77-86.
[38] Chablani L, Tawde S A, D'Souza M J. Spray-dried microparticles: a potential vehicle for oral delivery of vaccines. J Microencapsul. 2012; 29:388-97.
[39] Shastri P N, Ubale R V, D'Souza M J. Implementation of mixture design for formulation of albumin containing enteric-coated spray-dried microparticles. Drug development and industrial pharmacy. 2013; 39:164-75.
[40] Kim J Y, Choi W I, Kim Y H, Tae G. Brain-targeted delivery of protein using chitosan- and RVG peptide-conjugated, pluronic-based nano-carrier. Biomaterials. 2013; 34:1170-8.
[41] Golde W T, Gollobin P, Rodriguez L L. A rapid, simple, and humane method for submandibular bleeding of mice using a lancet. Lab animal. 2005; 34:39-43.
[42] Saraiva C, Praca C, Ferreira R, Santos T, Ferreira L, Bernardino L.

Nanoparticle-mediated brain drug delivery: Overcoming blood-brain barrier to treat neurodegenerative diseases. Journal of controlled release: official journal of the Controlled Release Society. 2016; 235:34-47.
[43] Etame A B, Smith C A, Chan W C, Rutka J T. Design and potential application of PEGylated gold nanoparticles with size-dependent permeation through brain microvasculature. Nanomedicine: nanotechnology, biology, and medicine. 2011; 7:992-1000.
[44] Hanada S, Fujioka K, Inoue Y, Kanaya F, Manome Y, Yamamoto K. Cell-based in vitro blood-brain barrier model can rapidly evaluate nanoparticles' brain permeability in association with particle size and surface modification. International journal of molecular sciences. 2014; 15:1812-25.
[45] Sonavane G, Tomoda K, Makino K. Biodistribution of colloidal gold nanoparticles after intravenous administration: effect of particle size. Colloids and surfaces B, Biointerfaces. 2008; 66:274-80.
[46] Zughaier S M, Shafer W M, Stephens D S. Antimicrobial peptides and endotoxin inhibit cytokine and nitric oxide release but amplify respiratory burst response in human and murine macrophages. Cellular microbiology. 2005; 7:1251-62.
[47] Bogdan C, Rollinghoff M, Diefenbach A. The role of nitric oxide in innate immunity. Immunological reviews. 2000; 173:17-26.
[48] Kumar P, Wu H, McBride J L, Jung K E, Kim M H, Davidson B L, et al. Transvascular delivery of small interfering RNA to the central nervous system. Nature. 2007; 448:39-43.
[49] Son S, Hwang D W, Singha K, Jeong J H, Park T G, Lee D S, et al. RVG peptide tethered bioreducible polyethylenimine for gene delivery to brain. Journal of controlled release: official journal of the Controlled Release Society. 2011; 155:18-25.
[50] Gooding M, Malhotra M, McCarthy D J, Godinho B M, Cryan J F, Darcy R, et al. Synthesis and characterization of rabies virus glycoprotein-tagged amphiphilic cyclodextrins for siRNA delivery in human glioblastoma cells: in vitro analysis. European journal of pharmaceutical sciences: official journal of the European Federation for Pharmaceutical Sciences. 2015; 71:80-92.
[51] Abbott N J, Patabendige A A, Dolman D E, Yusof S R, Begley D J. Structure and function of the blood-brain barrier. Neurobiology of disease. 2010; 37:13-25.
[52] Ballabh P, Braun A, Nedergaard M. The blood-brain barrier: an overview: structure, regulation, and clinical implications. Neurobiology of disease. 2004; 16:1-13.
[53] Kulkarni S A, Feng S S. Effects of particle size and surface modification on cellular uptake and biodistribution of polymeric nanoparticles for drug delivery. Pharmaceutical research. 2013; 30:2512-22.
[54] Kou L, Sun J, Zhai Y, He Z. The endocytosis and intracellular fate of nanomedicines: Implication for rational design. Asian Journal of Pharmaceutical Sciences. 2013; 8:1-10.
[55] Yeung T, Gilbert G E, Shi J, Silvius J, Kapus A, Grinstein S. Membrane phosphatidylserine regulates surface charge and protein localization. Science (New York, N.Y. 2008; 319:210-3.
[56] Tan J, Shah S, Thomas A, Ou-Yang H D, Liu Y. The influence of size, shape and vessel geometry on nanoparticle distribution. Microfluidics and nanofluidics. 2013; 14:77-87.
[57] Truong N P, Whittaker M R, Mak C W, Davis T P. The importance of nanoparticle shape in cancer drug delivery. Expert opinion on drug delivery. 2015; 12:129-42.
Abbott N J, Patabendige A A, Dolman D E, Yusof S R, Begley D J (2010) Structure and function of the blood-brain barrier. Neurobiology of disease 37: 13-25.
Alimohammadi H, Silver W L (2000) Evidence for nicotinic acetylcholine receptors on nasal trigeminal nerve endings of the rat. Chemical senses 25: 61-6.
Anand U, Parikh A, Ugwu M C, Agu R U (2014) Drug transporters in the nasal epithelium: an overview of strategies in targeted drug delivery. Future medicinal chemistry 6: 1381-97.
Ballabh P, Braun A, Nedergaard M (2004) The blood-brain barrier: an overview: structure, regulation, and clinical implications. Neurobiology of disease 16: 1-13.
Blesch A, Tuszynski M H (2007) Transient growth factor delivery sustains regenerated axons after spinal cord injury. J Neurosci 27: 10535-45.
Born J, Lange T, Kern W, McGregor G P, Bickel U, Fehm H L (2002) Sniffing neuropeptides: a transnasal approach to the human brain. Nat Neurosci 5: 514-6.
Broadwell R D, Baker-Cairns B J, Friden P M, Oliver C, Villegas J C (1996) Transcytosis of protein through the mammalian cerebral epithelium and endothelium. III. Receptor-mediated transcytosis through the blood-brain barrier of blood-borne transferrin and antibody against the transferrin receptor. Experimental neurology 142: 47-65.
Broderick D F, Schweitzer K J, Wszolek Z K (2009) Vascular risk factors and dementia: how to move forward? Neurology 73: 1934-5.
Carson D S, Guastella A J, Taylor E R, McGregor I S (2013) A brief history of oxytocin and its role in modulating psychostimulant effects. Journal of psychopharmacology (Oxford, England) 27: 231-47.
Curry D W, Young M B, Tran A N, Daoud G E, Howell L L (2018) Separating the agony from ecstasy: R(−)-3,4-methylenedioxymethamphetamine has prosocial and therapeutic-like effects without signs of neurotoxicity in mice. Neuropharmacology 128: 196-206.
Dal Monte O, Noble P L, Turchi J, Cummins A, Averbeck B B (2014) CSF and blood oxytocin concentration changes following intranasal delivery in macaque. PLoS One 9: e103677.
Dawbarn D, Allen S J (2003) Neurotrophins and neurodegeneration. Neuropathology and applied neurobiology 29: 211-30.
Donaldson Z R, Young L J (2008) Oxytocin, vasopressin, and the neurogenetics of sociality. Science (New York, N.Y. 322: 900-4.
Dumont G J, Sweep F C, van der Steen R, Hermsen R, Donders A R, Touw D J, van Gerven J M, Buitelaar J K, Verkes R J (2009) Increased oxytocin concentrations and prosocial feelings in humans after ecstasy (3,4-methylenedioxymethamphetamine) administration. Soc Neurosci 4: 359-66.
Engelmann M, Wotjak C T, Neumann I, Ludwig M, Landgraf R (1996) Behavioral consequences of intracerebral vasopressin and oxytocin: focus on learning and memory. Neurosci Biobehav Rev 20: 341-58.
Gan C W, Feng S S (2010) Transferrin-conjugated nanoparticles of poly(lactide)-D-alpha-tocopheryl polyethylene glycol succinate diblock copolymer for targeted drug delivery across the blood-brain barrier. Biomaterials 31: 7748-57.
Harris D S, Baggott M, Mendelson J H, Mendelson J E, Jones R T (2002) Subjective and hormonal effects of 3,4-methylenedioxymethamphetamine (MDMA) in humans. Psychopharmacology (Berl) 162: 396-405.
He F, Yin F, Peng J, Li K Z, Wu L W, Deng X L (2010) [Immortalized mouse brain endothelial cell line Bend.3 displays the comparative barrier characteristics as the primary brain microvascular endothelial cells]. Zhongguo dang dai er ke za zhi=Chinese journal of contemporary pediatrics 12: 474-8.
Herrmann N, Chau S A, Kircanski I, Lanctot K L (2011) Current and emerging drug treatment options for Alzheimer's disease: a systematic review. Drugs 71: 2031-65.
Jones A K, Bejugam N K, Nettey H, Addo R, D'Souza M J (2011) Spray-dried doxorubicin-albumin microparticulate systems for treatment of multidrug resistant melanomas. Journal of drug targeting 19: 427-33.
Keech B, Crowe S, Hocking D R (2018) Intranasal oxytocin, social cognition and neurodevelopmental disorders: A meta-analysis. Psychoneuroendocrinology 87: 9-19.
Keiger C J, Case L D, Kendal-Reed M, Jones K R, Drake A F, Walker J C (2003) Nicotinic cholinergic receptor expression in the human nasal mucosa. The Annals of otology, rhinology, and laryngology 112: 77-84.
Kim J Y, Choi W I, Kim Y H, Tae G (2013) Brain-targeted delivery of protein using chitosan- and RVG peptide-conjugated, pluronic-based nano-carrier. Biomaterials 34: 1170-8.
Klemp K, Woldbye D P (2001) Repeated inhibitory effects of NPY on hippocampal CA3 seizures and wet dog shakes. Peptides 22: 523-7.
Kolluru L P, Rizvi S A, D'Souza M, D'Souza M J (2013) Formulation development of albumin based theragnostic nanoparticles as a potential delivery system for tumor targeting. Journal of drug targeting 21: 77-86.
Lafay F, Coulon P, Astic L, Saucier D, Riche D, Holley A, Flamand A (1991) Spread of the CVS strain of rabies virus and of the avirulent mutant AvOl along the olfactory pathways of the mouse after intranasal inoculation. Virology 183: 320-30.
Leng G, Ludwig M (2016) Intranasal Oxytocin: Myths and Delusions. Biol Psychiatry 79: 243-50.
Li H, Sheppard D N, Hug M J (2004) Transepithelial electrical measurements with the Ussing chamber. Journal of cystic fibrosis: official journal of the European Cystic Fibrosis Society 3 Suppl 2: 123-6.
Li Y, Ogris M, Wagner E, Pelisek J, Ruffer M (2003) Nanoparticles bearing polyethyleneglycol-coupled transferrin as gene carriers: preparation and in vitro evaluation. International journal of pharmaceutics 259: 93-101.
Liu L, Duff K (2008) A technique for serial collection of cerebrospinal fluid from the cisterna magna in mouse. Journal of visualized experiments: JoVE.
Liu Y, An S, Li J, Kuang Y, He X, Guo Y, Ma H, Zhang Y, Ji B, Jiang C (2016) Brain-targeted co-delivery of therapeutic gene and peptide by multifunctional nanoparticles in Alzheimer's disease mice. Biomaterials 80: 33-45.
Makadia H K, Siegel S J (2011) Poly Lactic-co-Glycolic Acid (PLGA) as Biodegradable Controlled Drug Delivery Carrier. Polymers 3: 1377-1397.
Mann J F, Stieh D, Klein K, de Stegmann D S, Cranage M P, Shattock R J, McKay P F (2012) Transferrin conjugation confers mucosal molecular targeting to a model HIV-1 trimeric gp140 vaccine antigen. Journal of controlled release: official journal of the Controlled Release Society 158: 240-9.
Misra A, Ganesh S, Shahiwala A, Shah S P (2003) Drug delivery to the central nervous system: a review. Journal of pharmacy & pharmaceutical sciences: a publication of the Canadian Society for Pharmaceutical Sciences, Societe canadienne des sciences pharmaceutiques 6: 252-73.
Modi M E, Connor-Stroud F, Landgraf R, Young L J, Parr L A (2014) Aerosolized oxytocin increases cerebrospinal fluid oxytocin in rhesus macaques. Psychoneuroendocrinology 45: 49-57.
Modi M E, Young L J (2012) The oxytocin system in drug discovery for autism:
   animal models and novel therapeutic strategies. Horm Behav 61: 340-50.
Montesano R, Pepper M S, Mohle-Steinlein U, Risau W, Wagner E F, Orci L (1990) Increased proteolytic activity is responsible for the aberrant morphogenetic behavior of endothelial cells expressing the middle T oncogene. Cell 62: 435-45.
Morley K C, Arnold J C, McGregor I S (2005) Serotonin (1A) receptor involvement in acute 3,4-methylenedioxymethamphetamine (MDMA) facilitation of social interaction in the rat. Prog Neuropsychopharmacol Biol Psychiatry 29: 648-57.
Murnane K S, Fantegrossi W E, Godfrey J R, Banks M L, Howell L L (2010) Endocrine and neurochemical effects of 3,4-methylenedioxymethamphetamine and its stereoisomers in rhesus monkeys. J Pharmacol Exp Ther 334: 642-50.
Murnane K S, Kimmel H L, Rice K C, Howell L L (2012) The neuropharmacology of prolactin secretion elicited by 3,4-methylenedioxymethamphetamine (“ecstasy”): a concurrent microdialysis and plasma analysis study. Horm Behav 61: 181-90.
Nagahara A H, Merrill D A, Coppola G, Tsukada S, Schroeder B E, Shaked G M, Wang L, Blesch A, Kim A, Conner J M, Rockenstein E, Chao M V, Koo E H, Geschwind D, Masliah E, Chiba A A, Tuszynski M H (2009) Neuroprotective effects of brain-derived neurotrophic factor in rodent and primate models of Alzheimer's disease. Nature medicine 15: 331-7.
O'Brien R J, Wong P C (2011) Amyloid precursor protein processing and Alzheimer's disease. Annu Rev Neurosci 34: 185-204.
Oettinger C W, D'Souza M J, Akhavein N, Peer G T, Taylor F B, Kinasewitz G T (2007) Pro-inflammatory cytokine inhibition in the primate using microencapsulated antisense oligomers to N F-kappaB. J Microencapsul 24: 337-48.
Opal S M, DePalo V A (2000) Anti-inflammatory cytokines. Chest 117: 1162-72.
Pan X, Guan J, Yoo J W, Epstein A J, Lee L J, Lee R J (2008) Cationic lipid-coated magnetic nanoparticles associated with transferrin for gene delivery. International journal of pharmaceutics 358: 263-70.
Pang Z, Gao H, Yu Y, Guo L, Chen J, Pan S, Ren J, Wen Z, Jiang X (2011) Enhanced intracellular delivery and chemotherapy for glioma rats by transferrin-conjugated biodegradable polymersomes loaded with doxorubicin. Bioconjugate chemistry 22: 1171-80.
Pardridge W M (2014) Blood-brain barrier drug delivery of IgG fusion proteins with a transferrin receptor monoclonal antibody. Expert opinion on drug delivery: 1-16.
Qian Z M, Li H, Sun H, Ho K (2002) Targeted drug delivery via the transferrin receptor-mediated endocytosis pathway. Pharmacol Rev 54: 561-87.
Reibel S, Nadi S, Benmaamar R, Larmet Y, Carnahan J, Marescaux C, Depaulis A (2001) Neuropeptide Y and epilepsy: varying effects according to seizure type and receptor activation. Peptides 22: 529-39.
Robertson G L, Klein L A, Roth J, Gorden P (1970) Immunoassay of plasma vasopressin in man. Proc Natl Acad Sci USA 66: 1298-305.
Sahoo S K, Labhasetwar V (2005) Enhanced antiproliferative activity of transferrin-conjugated paclitaxel-loaded nanoparticles is mediated via sustained intracellular drug retention. Molecular pharmaceutics 2: 373-83.
Sikorski E E, Hallmann R, Berg E L, Butcher E C (1993) The Peyer's patch high endothelial receptor for lymphocytes, the mucosal vascular addressin, is induced on a murine endothelial cell line by tumor necrosis factor-alpha and IL-1. Journal of immunology (Baltimore, Md.: 1950) 151: 5239-50.
Sloane E, Ledeboer A, Seibert W, Coats B, van Strien M, Maier S F, Johnson K W, Chavez R, Watkins L R, Leinwand L, Milligan E D, Van Dam A M (2009) Anti-inflammatory cytokine gene therapy decreases sensory and motor dysfunction in experimental Multiple Sclerosis: MOG-EAE behavioral and anatomical symptom treatment with cytokine gene therapy. Brain, behavior, and immunity 23: 92-100.
Soderquist R G, Mahoney M J (2010) Central nervous system delivery of large molecules: challenges and new frontiers for intrathecally administered therapeutics. Expert opinion on drug delivery 7: 285-93.
Srinivasan B, Kolli A R, Esch M B, Abaci H E, Shuler M L, Hickman J J (2015) TEER measurement techniques for in vitro barrier model systems. Journal of laboratory automation 20: 107-26.
Strecker K, Schwarz J (2008) Parkinson's disease: emerging pharmacotherapy. Expert opinion on emerging drugs 13: 573-91.
Striepens N, Kendrick K M, Hanking V, Landgraf R, Wullner U, Maier W, Hurlemann R (2013) Elevated cerebrospinal fluid and blood concentrations of oxytocin following its intranasal administration in humans. Scientific reports 3: 3440.
Sundaram S, Roy S K, Ambati B K, Kompella U B (2009) Surface-functionalized nanoparticles for targeted gene delivery across nasal respiratory epithelium. Faseb j 23: 3752-65.
Terryn S, Francart A, Lamoral S, Hultberg A, Rommelaere H, Wittelsberger A, Callewaert F, Stohr T, Meerschaert K, Ottevaere I, Stortelers C, Vanlandschoot P, Kalai M, Van Gucht S (2014) Protective effect of different anti-rabies virus VHH constructs against rabies disease in mice. PLoS One 9: e109367.
Thompson M R, Callaghan P D, Hunt G E, Cornish J L, McGregor I S (2007) A role for oxytocin and 5-H T(1A) receptors in the prosocial effects of 3,4 methylenedioxymethamphetamine (“ecstasy”). Neuroscience 146: 509-14.
Tong W, Wang L, D'Souza M J (2003) Evaluation of PLGA microspheres as delivery system for antitumor agent-camptothecin. Drug development and industrial pharmacy 29: 745-56.
Ulbrich K, Hekmatara T, Herbert E, Kreuter J (2009) Transferrin- and transferrin-receptor-antibody-modified nanoparticles enable drug delivery across the blood-brain barrier (BBB). European journal of pharmaceutics and biopharmaceutics: official journal of Arbeitsgemeinschaft fur Pharmazeutische Verfahrenstechnik eV 71: 251-6.
Walker D, Lue L F (2007) Anti-inflammatory and immune therapy for Alzheimer's disease: current status and future directions. Current neuropharmacology 5: 232-43.
Watanabe T, Dohgu S, Takata F, Nishioku T, Nakashima A, Futagami K, Yamauchi A, Kataoka Y (2013) Paracellular barrier and tight junction protein expression in the immortalized brain endothelial cell lines bEND.3, bEND.5 and mouse brain endothelial cell 4. Biological & pharmaceutical bulletin 36: 492-5.
Wiley D T, Webster P, Gale A, Davis M E (2013) Transcytosis and brain uptake of transferrin-containing nanoparticles by tuning avidity to transferrin receptor. Proc Natl Acad Sci USA 110: 8662-7.
Williams R L, Courtneidge S A, Wagner E F (1988) Embryonic lethalities and endothelial tumors in chimeric mice expressing polyoma virus middle T oncogene. Cell 52: 121-31.
Woldbye D P (1998) Antiepileptic effects of NPY on pentylenetetrazole seizures. Regulatory peptides 75-76: 279-82.
Wuest D M, Wing A M, Lee K H (2013) Membrane configuration optimization for a murine in vitro blood-brain barrier model. J Neurosci Methods 212: 211-21.
Yan F, Wang Y, He S, Ku S, Gu W, Ye L (2013) Transferrin-conjugated, fluorescein-loaded magnetic nanoparticles for targeted delivery across the blood-brain barrier. Journal of materials science Materials in medicine 24: 2371-9.
Ying X, Wen H, Lu W L, Du J, Guo J, Tian W, Men Y, Zhang Y, Li R J, Yang T Y, Shang D W, Lou J N, Zhang L R, Zhang Q (2010) Dual-targeting daunorubicin liposomes improve the therapeutic efficacy of brain glioma in animals. Journal of controlled release: official journal of the Controlled Release Society 141: 183-92.
Zhang X, Koh C G, Yu B, Liu S, Piao L, Marcucci G, Lee R J, Lee L J (2009) Transferrin receptor targeted lipopolyplexes for delivery of antisense oligonucleotide g3139 in a murine k562 xenograft model. Pharmaceutical research 26: 1516-24.

What is claimed is:

1. A composition comprising:
a) an active material;
b) a nanoparticle, wherein the nanoparticle encapsulates the active material; and
c) at least one targeting ligand, wherein the targeting ligand is coupled to the nanoparticle.

2. The composition of claim 1, wherein the active material is a neuropeptide.

3. The composition of claim 2, wherein the active material is oxytocin.

4. The composition of claim 1, wherein the nanoparticle is comprised of BSA.

5. The composition of claim 1, wherein the at least one targeting ligand is RVG.

6. The composition of claim 5, wherein the RVG is adsorbed onto the nanoparticle.

7. A method of treating a neurological disorder comprising administering the composition of claim 1 to a patient.

8. The method of claim 7, wherein the neurological disorder is a central nervous system disorder.

9. The method of claim 7, wherein the neurological disorder is Autism Spectrum disorder.

10. The method of claim 9, wherein the composition of claim 1 is administered intranasally.
